SEQUENCE LISTING (8 sequences)

SEQ ID NO: 1 - DNA, 20 bp, Artificial Sequence
  Primer
gggatgatgacgacctgcta

SEQ ID NO: 2 - DNA, 20 bp, Artificial Sequence
  Primer
tgtcgttgcttgtctctcct

SEQ ID NO: 3 - DNA, 20 bp, Artificial Sequence
  Primer
tgcagagacacagacagagg

SEQ ID NO: 4 - DNA, 20 bp, Artificial Sequence
  Primer
gccagtgaatgagtagcagc

SEQ ID NO: 5 - DNA, 19 bp, Artificial Sequence
  Primer
actcttccagccttccttc

SEQ ID NO: 6 - DNA, 20 bp, Artificial Sequence
  Primer
atctccttctgcatcctgtc

SEQ ID NO: 7 - DNA, 20 bp, Artificial Sequence
  Primer
ggtagcactgttgggcatct

SEQ ID NO: 8 - DNA, 20 bp, Artificial Sequence
  Primer
gaccccacagagacaagctc

(19) United States
(12) Patent Application Publication
(10) Pub. No.: US 2023/0089949 A1
(43) Pub. Date: Mar. 23, 2023

(54) SMALL MOLECULES FOR TREATING AGE-RELATED RETINAL DISEASES

(71) Applicant: University of Pittsburgh - Of the Commonwealth System of Higher Education, Pittsburgh, PA (US)

(72) Inventors: Lori Birder, Pittsburgh, PA (US); Edwin Kerry Jackson, Pittsburgh, PA (US); Amanda Wolf-Johnston, Evans City, PA (US); José-Alain Sahel, Pittsburgh, PA (US); Yuanyuan Chen, Pittsburgh, PA (US)

(73) Assignee: University of Pittsburgh - Of the Commonwealth System of Higher Education, Pittsburgh, PA (US)

(21) Appl. No.: 17/799,546

(22) PCT Filed: Apr. 19, 2021
(86) PCT No.: PCT/US2021/027991

§ 371 Date: Aug. 12, 2022

Related U.S. Application Data
(60) Provisional application No. 63/012,627, filed on Apr. 20, 2020.

(51) Int. Cl. A61K 31/522; A61P 27/02

(52) U.S. Cl. CPC A61K 31/522; A61P 27/02

(57) ABSTRACT

Disclosed herein are methods of treating age-related retinal diseases by administering to the subject a therapeutically effective amount of a purine nucleoside phosphorylase (PNPase) inhibitor and/or a PNPase purine nucleoside substrate. In some examples, the subject can have AMD or glaucoma.

### CROSS REFERENCE TO RELATED APPLICATIONS

This claims the benefit of U.S. Provisional Application No. 63/012,627, filed Apr. 20, 2020, which is incorporated herein by reference.

### ACKNOWLEDGMENT OF GOVERNMENT SUPPORT

This invention was made with government support under grant number AG063753 awarded by the National Institutes of Health. The government has certain rights in the invention.

### FIELD

This relates to the field of purine nucleoside phosphorylase (PNPase) inhibitors and PNPase purine nucleoside substrates and their use for treating age-related retinal diseases.

### BACKGROUND

Eye disease and vision loss affects millions of people, many of which have onset of an eye disease as a function of age. In some cases, the affected individuals are older, but age-related disease can also manifest in individuals as young as children. Glaucoma is one of the world's leading causes of irreversible blindness and is characterized by the slow progressive degeneration of retinal ganglion cells and their axons. Retinal ganglion cells operate as the final stage in the phototransductive visual pathway of the retina, tasked with the projection of electrochemical information to the brain along their axons which make up the optic nerve. Retinal ganglion cells are irreplaceable, making their dysfunction and subsequent loss a severe detriment to vision and thus, quality of life. Age-related macular degeneration (AMD) is a disease that is a major cause of blindness in the United States and other industrialized nations. Early AMD is characterized clinically by drusen, which are extracellular deposits of proteins, lipids, and cellular debris, that are located beneath the retinal pigment epithelium. A need remains for treatments of age-related retinal diseases, such as, but not limited to, glaucoma and AMD.

### SUMMARY

Methods of treating age-related retinal diseases in a subject are disclosed herein. In some examples, the methods include selecting a subject with an age-related retinal disease. In some examples, the methods include administering to the subject a therapeutically effective amount of a purine nucleoside phosphorylase (PNPase) inhibitor or a PNPase purine nucleoside substrate, thereby treating the age-related retinal disease. Methods are also disclosed for reducing inflammation and improving or restoring vasculature in a retina of a subject in need thereof. These methods include selecting the subject in need of reduced inflammation and/or improved vasculature in the retina, and administering to the subject a therapeutically effective amount of a PNPase inhibitor and/or a PNPase purine nucleoside substrate.

In specific, non-limiting examples, subject has AMD, ganglion cell degeneration, glaucoma, Leber congenital amaurosis (LCA), retinitis pigmentosa (RP), cone rod dystrophy, retinal detachment, hypertensive retinopathy, retinal vein occlusion (RVO), central retinal artery occlusion (CRAO), branch retinal artery occlusion (BRAO), or diabetic retinopathy.

The foregoing and other objects and features of the disclosure will become more apparent from the following detailed description, which proceeds with reference to the accompanying figures.

### BRIEF DESCRIPTION OF THE DRAWINGS

FIG. 1 illustrates the anatomy of the eye with and without age-related macular degeneration.

FIG. 2 shows a set of photographs illustrating mitochondrial dysfunction consistent with dry eye AMD induced by the environmental toxicant hydroquinone.

FIG. 3 shows a schematic diagram illustrating how oxidative stress (and decreased mitochondrial function) play a role in AMD pathogenesis and progression. Increased reactive oxygen species (ROS) formation and decreased mitochondrial function lowers ATP synthesis and affects retinal pigment epithelium function. PNPase inhibitors such as 8-aminoguanine (8-AG) will reduce oxidative stress to increase mitochondrial function and treat AMD.

FIG. 4 is a schematic diagram illustrating macular fibrosis in neovascular AMD, which may be due in part to persistent damage to retinal pigment epithelium (RPE) and outer layers of the neuronal retina. It is believed that PNPase inhibitors such as 8-aminoguanine (8-AG) will have a restorative effect on macular fibrosis and cellular health to reduce AMD.

FIG. 5 is a schematic diagram illustrating vascular dysfunction in age-related macular degeneration, which is associated with loss of vascular density (decreased branching), thinning or constriction of existing blood vessels, structural consequences (cell loss/apoptosis), and/or neovascularization (increased VEGF) and increased vascular permeability. It is believed that PNPase inhibitors such as 8-AG will have a restorative effect on vascular health to reduce AMD.

FIG. 6 is a schematic diagram illustrating a proposed mechanism of action for 8-AG based treatment of age-related macular degeneration, by inhibiting production of reactive oxygen species and corresponding oxidative stress.

FIG. 7 is a schematic diagram further illustrating a proposed mechanism of action for 8-AG based treatment of age-related macular degeneration, by reducing ‘damaging’ PNPase products (for example, hypoxanthine which is a source of a ROS) and increasing protective PNPase substrates (for example, inosine and guanosine).

FIG. 8 shows histology of the outer nuclear layer (ONL) in Fischer 344 rats at different ages and with and without 8-AG treatment. With advance age, there is likely to be a loss of photoreceptors with associated thinning and eventual loss of the overlying ONL, a process that was reversed by 8-AG treatment.

FIGS. 9A-9B show ONL thickness in young rats and aged rats with and without 8-AG treatment. FIG. 9A shows the locations of rat retina, where the ONL thickness was measured. FIG. 9B shows the thickness based on location in the retina for the examined rat retina. Quantification of ONL thickness confirms that 8-AG treatment significantly protects photoreceptors.

FIG. 10 presents an overview of the role of purine nucleoside phosphorylase (PNPase) in purine degradation. 8-Aminoguanine (8-AG), by inhibiting PNPase, may upregulate tissue-protective purines and reduce tissue-damaging HX and xanthine that generates reactive oxygen species (ROS). ADA, adenosine deaminase; XO, xanthine oxidase/dehydrogenase; GAH, guanine aminohydrolase/deaminase. This figure shows the potential mechanism of action of 8-AG in restoring purine balance.

FIGS. 11A-11D show that 8-AG protects the photoreceptors in the aged F344 rat retinae from degeneration. A. H&E staining and immunofluorescence of rhodopsin (RHO) with young (3 months (m)), aged (24 m) and 8-AG treated aged retinae. White lines indicate the outer/inner segments (OS/IS, the primary ciliary structure of photoreceptor cells) and. Outer nuclear layer (ONL, the layer containing nuclei of photoreceptors), demonstrating these layers are thinner in the aged versus young retinae, and thicker in the 8-AG treated aged retinae versus untreated control. B, C and D are spidergrams of ONL, OS/IS thickness and rhodopsin intensity in the young, aged and 8-AG treated retinae, shown with circles in black, grey and dark grey, respectively. N=8, P values were calculated by two-way analysis of variants analysis (ANOVA). IP, IE and IC are inferior peripheral, equatorial, central retinal positions, and SC, SE and SP are superior peripheral, equatorial and central retinal positions. This figure demonstrates the strong protection by oral 8-AG in a rat model of age-related retinal degeneration.

FIGS. 12A-12F. illustrate that 8-AG protects Rho^{P23H/+} knock-in mouse retina from degeneration. A-C. H&E staining of retinal sections from wild-type (WT) and Rho^{P23H/+} mice that were treated with PBS or 8-AG for 5 weeks (wks), starting at postnatal day 15, respectively. D. Spider gram of ONL thickness. P1 was calculated by a two-way ANOVA. E and F. Scotopic a- and b-wave responses from mice at PND50, respectively. *, **, ***, ****, P<0.05, 0.01, 0.001, and 0.0001 respectively, by a one-way ANOVA. WT, PBS treated Rho^{P23H/+} and 8-AG treated Rho^{P23H/+} mice retinae were labeled in black squares, grey circles, and dark grey triangles, respectively. This figure shows the strong retinal protection by systemic administration of 8-AG in a mouse model of retinitis pigmentosa.

FIGS. 13A-13E illustrate that the expression of enzymes in the purine degradation pathways is dysregulated in the degenerative retinae. Fold change of transcripts from Rho^{P23H/+} vs. WT mouse retinae at 1, 3 and 6 months of age. A. Pnp, expressing PNPase. B. Xdh, expressing xanthine dehydrogenase/oxidase. C. Gda, expressing guanine deaminase. D. Adat1, expressing adenosine deaminase, tRNA specific1. N=3. Color of each dot is coded for the P value by a one-way ANOVA. E. Upregulation of Pnp, Xdh and Gda will lead to accumulation of xanthine, and down regulation of Adat1 will lead to reduction of inosine. This figure shows the expression of genes involved in purine salvage pathway is dysregulated in the mouse model of retinitis pigmentosa, indicating increased production of tissue damaging purines and reduced production of tissue-protective purines.

FIG. 14A-14L evidence that purine metabolism is altered in the aged F344 rat retinae. Old (22 m) and young (3 m) F344 rat retinae were isolated and purine metabolome was analyzed by UPLC-MS/MS. A-L, bar plots of purines per retina. Young, black; old, shaded. Bars and errors are mean±standard deviation (SD). N=4, and P<0.05 were shown in value, calculated by a two-tail unequivocal Student's T-test. This figure shows that the purine metabolites are changed in the retinae of the rat model of age-related retinal degeneration, which may contribute to photoreceptor cell death.

FIGS. 15A-15D document that 8-AG reverses age-associated biomarkers of cell stress to a younger state. A-D, immunoblot of MFN2, SOD2, uncleaved and cleaved caspase 3 (CASP3) and Rhodopsin in the young (3 m), aged (24 m) and 8-AG treated (24 m) F344 rat retinae, respectively. Total protein blots were shown as a loading control. Normalized band intensities were plotted in each graph. Bars and errors are mean±SD. All samples were run on the same blot; representative samples were not contiguous. N=4-8, and *,***, P<0.05, 0.001 were shown in value, calculated by a two-tail unequivocal Student's T-test. This figure shows the levels of proteins involved in mitochondria function, response to oxidative stress, cell death and phototransduction are dysregulated in the degenerative retinae, which are restored by 8-AG treatment.

FIGS. 16A-16D evidence that 8AG reduced macrophage expression, without affecting Müller glial activation in aged F344 rats. A and C, Immunofluorescence of glial fibrillary acidic protein (GFAP) and cluster of differentiation (CD)68, markers of activated Müller glia and macrophages, in the retinae cross-sections of young (3 m), aged (24 m) and aged rats treated with 8AG, respectively. 8AG was administered in drinking water at 5 mg/kg bw per day for eight weeks starting at 22 months of age. Arrowheads, CD68+ cells in the retinae, suggesting 8-AG reduced macrophages in the aged retinae. B and D, spidergrams of GFAP intensity and number of CD68+ cells at central, equatorial and peripheral retinae of young, aged and aged rats treated with 8AG, shown in circles, squares and triangles, respectively. N=4. ***, P₁<0.001 by a Two-way ANOVA. Arrow heads, CD68+ cells in the retinae. This figure shows that oral 8-AG reduced the number of activated immune cells in the aged-retinae, suggesting the anti-inflammatory effects that contribute to retinal protection.

FIGS. 17A-17F show that spectral domain optical coherence tomography (SD-OCT) scanning indicated 8-AG increases ONL thickness, compared to pretreatment levels. Aged F344 rats were treated with 8-AG in drinking water (5 mg/kg body weight (bw) per day) starting at 24 months of age, and SD-OCT was taken at 0 and 4 wks. A & B and D & E are OCT scanning images of an untreated retina and an 8-AG treated retina at 0 and 4 wks at central and temporial peripheral regions at top and bottom panels, respectively. C and F, spidergrams of ONL thickness in the untreated and 8-AG treated aged rat retinae, respectively. Black and red are measurements at 0 and 4 wks of treatment. N=12. Data and error bars are means and SEM. Arrow heads, areas that showed morphological changes with time. This figure shows that 8-AG not only prevent photoreceptors from degeneration, but it also restores the morphology of photoreceptors that is declined in the rat model of age-related retinal degeneration.

FIGS. 18A-18I show that the scotopic electroretinogram (ERG) responses of aged F344 rats were increased after 4 wks of 8AG treatment. A & B, D & E, and G & H are multi-flash scotopic a-wave, scotopic b-wave, and photopic b-wave electroretinogram (ERG) responses of untreated and 8-AG treated aged F344 rats that were recorded at 0 and 4 weeks of treatment, respectively. N=12. Data and error bars are means and SEM. * and ***, P<0.05 and <0.001 by a two-way ANOVA. C, F and I are fold changes of individual aged rat eye's scotopic a-, b- and photopic b waves at 10 cd.s/m2 flash intensity at 4 wks vs. 0 wk time points, respectively. Circles and triangles are from untreated and 8-AG treated aged rats, respectively. * and **, P<0.05 and <0.01 by a two-tailed unpaired Student's T-test. This figure shows 8-AG increased retinal function with time, indicating a functional restoration instead of preservation.

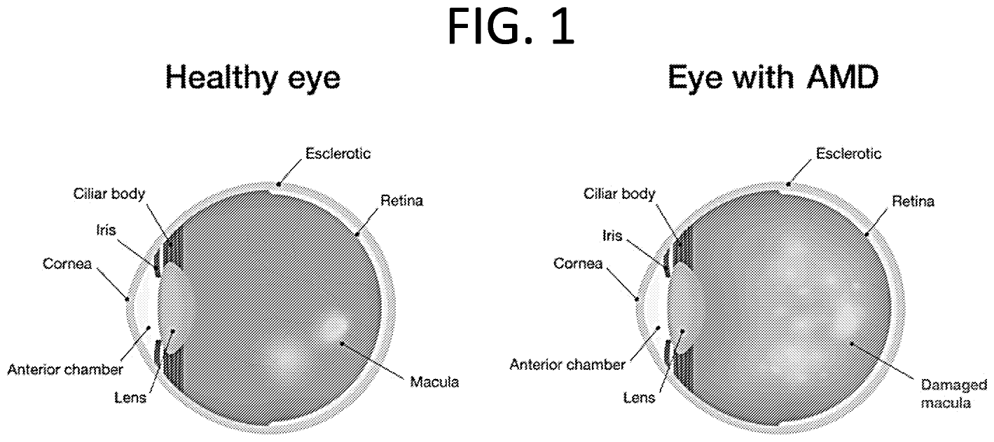

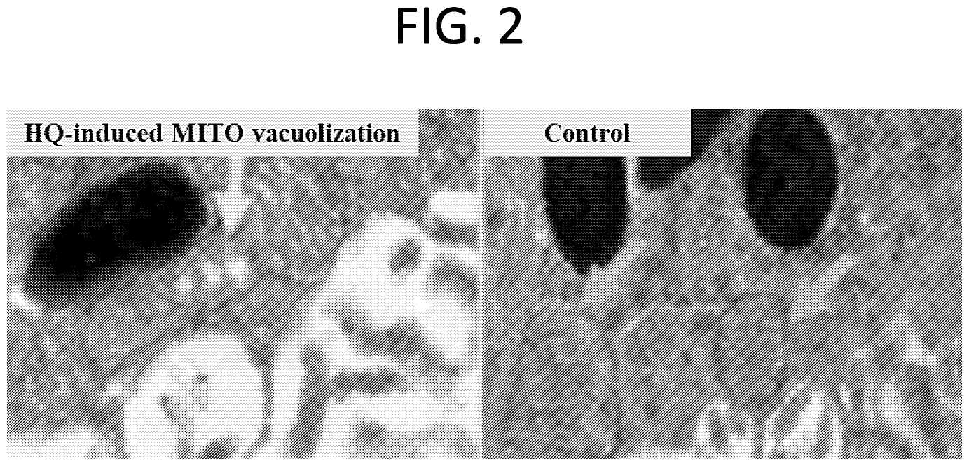

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file [Sequence_Listing, Apr. 19, 2021, size of 1.82 KB], which is incorporated by reference herein. In the accompanying sequence listing:

SEQ ID NOs: 1-8 are the nucleic acid sequences of primers.

### DETAILED DESCRIPTION

Peroxynitrite (ONOO−) is formed in vivo from the diffusion-controlled reaction between superoxide anion (O₂⁻) and nitric oxide (NO) (Carballal S et al., Biochimica et Biophysica Acta, 1840:768-780, 2014). Further, ONOO− is a highly reactive nitrogen species (RNS) that can mediate nitration (such as insertion of —NO₂) of numerous endogenous compounds, including those containing a guanine moiety (Ohshima H et al., Antioxidants & Redox Signaling, 8:1033-1045, 2006; Szabo C et al., Nitric Oxide, 1:373-385, 1997; Yermilov V et al., FEBS Lett, 376:207-210, 1995). In this regard, ONOO− nitrates guanine moieties at position 8 of the purine ring to produce 8-nitroguanine units in DNA, RNA, and the guanine nucleotide pool (Ohshima H et al., Antioxidants & Redox Signaling, 8:1033-1045, 2006; Szabo C et al., Nitric Oxide, 1:373-385, 1997; Yermilov V et al., FEBS Lett, 376:207-210, 1995). Free guanine may be nitrated at the 8 position. In addition to RNS-mediated modification of guanine-containing compounds, reactive oxygen species (ROS), such as O₂⁻, can also modify position 8 of guanine moieties by inserting a hydroxyl functional group (Szabo C et al., Nitric Oxide, 1:373-385, 1997; Misiaszek R et al., Journal of Biological Chemistry, 279:32106-32115, 2004).

After modification of guanine moieties by RNS or ROS, subsequent catabolism of RNA, DNA, and the guanine nucleotide pool will release 8-nitroguanosine, 8-nitro-2-deoxyguanosine, 8-hydroxyguanosine, and 8-hydroxy-2-deoxyguanosine. Reduction of 8-nitro groups could yield 8-aminoguanosine and 8-amino-2-deoxyguanosine, and PNPase can convert such compounds into 8-aminoguanine (8-AG) (Osborne W R et al., Immunology, 59:63-67, 1986). In addition, PNPase may convert 8-nitroguanosine and 8-nitro-2-deoxyguanosine into 8-nitroguanine, and reduction of 8-nitroguanine would yield 8-AG. Similarly, PNPase may produce 8-hydroxyguanine from 8-hydroxyguanosine or 8-hydroxy-2-deoxyguanosine. Consistent with this framework are studies confirming the presence of 8-nitroguanosine, 8-aminoguanosine, 8-AG, 8-hydroxyguanosine, 8-nitroguanine, 8-hydroxyguanine, and 8-hydroxy-2-deoxyguanosine in tissues or urine (Akaike T et al., Proc Natl Acad Sci USA, 100:685-690, 2003; Sodum R S et al., Chem Res Toxicol, 6:269-276, 1993; Park E M et al., Proc Natl Acad Sci USA, 89:3375-3379, 1992; Ohshima H et al., Antioxid Redox Signal, 8:1033-1045, 2006; Fraga C G et al., Proc Natl Acad Sci USA, 87:4533-4537, 1990; Lam P M et al., Free Radic Biol Med, 52:2057-2063, 2012).

PNPase transition state analogs are also of use in the disclosed methods. In some non-limiting examples, the transition state analog can be forodesine or a derivative thereof (such as DADMe-immucillin-H, DATMe-immucillin-H, or SerMe-immucillin-H) or pharmaceutically acceptable salt thereof (such as a PNPase transition state analog chloride salt), or as described in U.S. Pat. Nos. 4,985,433; 4,985,434, 5,008,265; 5,008,270; 5,565,463; and 5,721,240 as well as US Published Patent Application No. 2018/0258091A1, all of which are incorporated herein by reference in their entireties. Pharmaceutically acceptable salts of these compounds are also of use.

Methods are disclosed herein that utilize PNPase inhibitors. In some embodiments, these methods include selecting a subject with an age-related retinal disease, and treating the subject with a therapeutically effective amount of a PNPase inhibitor or a PNPase purine nucleoside substrate, thereby treating the age-related retinal disease. In other embodiments, methods are disclosed for reducing inflammation and improving or restoring vasculature in a retina of a subject in need thereof. These methods include selecting the subject in need of reduced inflammation and/or improved vasculature in the retina, and administering to the subject a therapeutically effective amount of a PNPase inhibitor and/or a PNPase purine nucleoside substrate.

In some embodiments, the PNPase inhibitor is a guanine that includes a substituent at the 8-position, a guanosine comprising a substituent at the 8-position, an inosine comprising a substituent at the 8-position, a hypoxanthine comprising a substituent at the 8-position, a PNPase transition state analog, or a pharmaceutically acceptable salt thereof. In some embodiments, the substituent is amine, hydroxyl, nitro, nitroso, alkoxy, carbonyl, halogen, carboxyl, ester, carbonate, amide, or haloaliphatic. In particular embodiments, the substituent is amine. In some embodiments PNPase inhibitor is the guanine comprising a substituent at the 8-position or the guanosine comprising a substituent at the 8-position. In particular embodiments, the guanine comprising a substituent at the 8-position is 8-AG. In some embodiments, the PNPase transition state analog is 7-[(2S,3S,4R,5R)-3,4-dihydroxy-5-(hydroxymethyl)pyrrolidin-2-yl]-3H,4H,5H-pyrrolo[3,2-d]pyrimidin-4-one; 7-(((3R,4R)-3-hydroxy-4-(hydroxymethyl)pyrrolidin-1-yl)methyl)-3H-pyrrolo[3,2-d]pyrimidin-4(5H)-one; 7-(((2R,3S)-1,3,4-trihydroxybutan-2-ylamino)methyl)-3H-pyrrolo[3,2-d]pyrimidin-4(5H)-one; 7-((1,3-dihydroxypropan-2-ylamino)methyl)-3H-pyrrolo[3,2-d]pyrimidin-4(5H)-one; or a pharmaceutically acceptable salt thereof (such as a chloride salt).

In some embodiments, the PNPase inhibitor and/or a PNPase purine nucleoside substrate is administered orally, intravenously, or into the eye (such as on the conjunctiva) of the subject. In particular embodiments, the administering includes delivering the PNPase inhibitor and/or the PNPase purine nucleoside substrate into the eye of the subject. In particular embodiments, the administering includes repeated delivering to eye the subject.

In some embodiments, the subject has AMD, ganglion cell degeneration, glaucoma, LCA, RP, cone rod dystrophy, retinal detachment, hypertensive retinopathy, RVO, CRAO, BRAO, or diabetic retinopathy. In particular embodiments, the subject has the AMD. In particular embodiments, the subject has glaucoma. In some embodiments, administering to the subject a therapeutically effective amount of a PNPase inhibitor or a PNPase purine nucleoside substrate produces a decrease in at least one inflammatory cytokine (such as interleukin 1 beta (IL-1beta) or monocyte chemoattractant protein-1 (MCP-1)). In some embodiments, the subject is a veterinary subject. In some embodiments, the subject is a human subject, such as a subject greater than 50 years of age or greater than 60 years of age.

### I. Terms

Unless otherwise noted, technical terms are used according to conventional usage. Definitions of common terms in molecular biology can be found in Krebs et al (Eds.), Lewin's Genes XII, published by Jones & Bartlett Publishers, 2017; and Meyers et al. (eds.), The Encyclopedia of Cell Biology and Molecular Medicine, published by Wiley-VCH in 16 volumes, 2008; and other similar references.

As used herein, the singular forms “a,” “an,” and “the” refer to both the singular as well as plural unless the context clearly indicates otherwise. Further, compositions of use in the methods herein can be used alone or in combination. Although many methods and materials similar or equivalent to those described herein can be used, particular suitable methods and materials are described below. The term “comprises” means “includes.” The term “about” means within five percent, unless otherwise indicated. All publications, patent applications, patents, and other references mentioned herein are incorporated by reference in their entirety. In case of conflict, the present specification, including explanations of terms, will control. In addition, the materials, methods, and examples are illustrative only and not intended to be limiting. To facilitate review of the various embodiments, the following explanations of terms are provided.

Administration: To provide or give a subject an agent (such as a PNPase transition state analog or a guanine, guanosine, inosine, or hypoxanthine comprising a substituent at the 8-position) by any effective route. Exemplary routes of administration include, but are not limited to, direct administration (such as via ocular delivery), topical administration (such as on the surface of the eye), oral, injection (such as subcutaneous, intramuscular, intradermal, intraperitoneal, intravenous, and intratumoral), sublingual, rectal, transdermal, intranasal, vaginal, and inhalation routes.

Age-related retinal diseases: Eye diseases in which symptoms worsen with age. In some subjects, onset can begin to occur at about age 40 but can also manifest at age 50, 60, or later. In some subjects, such as subjects with congenital defects, manifestation and progression can occur as early as childhood, for example, subjects with LCA, cone rod dystrophy, or retinitis pigmentosa. Examples of age-related retinal diseases include AMD, ganglion cell degeneration, glaucoma, LCA, RP, cone rod dystrophy, retinal detachment, hypertensive retinopathy, RVO, CRAO, BRAO, or diabetic retinopathy.

AMD: AMD is caused by damage to the macula of the retina. Onset of AMD may be asymptomatic, but AMD gradually worsens over time and generally results in blurred or no vision in the center of the visual field in one or both eyes. It can be hard to recognize faces, drive, read, or perform other activities of daily life, and visual hallucinations may also occur. AMD typically occurs in older people, such as subjects about 50 years and older. Genetic factors and smoking can play a role. Diagnosis includes a complete eye exam, and severity can range from early, intermediate, and late types, in which the late type can further include “dry” and “wet” forms.

Dry AMD occurs over time, wherein macular tissue thins and breaks down. Symptoms can include visual distortions, reduced central vision in one or both eyes, a need for brighter light for reading or close work, increased difficulty adapting to low light levels, increased blurriness of printed words, decreased intensity or brightness of colors, and difficulty recognizing faces. Dry AMD is diagnosed by examining the back of the eye for drusen; testing for defects in the center of the vision (such a using an Amsler grid to identify whether straight lines in the grid to look faded, broken, or distorted, indicating the presence of dry AMD); fluorescein or indocyanine green angiography (examining for abnormal blood vessel or retinal changes); and/or optical coherence tomography (examining for retinal thinning, thickening, or swelling). Treatment can include rehabilitation for adapting to the loss of central vision (low vision rehabilitation) and implanting a telescopic lens.

Wet AMD follows dry AMD and includes abnormal blood vessel growth as well as fluid build up in the back of the eye, which can produce a bump in the macula, causing vision loss of distortion. In addition to the symptoms of dry AMD, wet AMD symptoms can also include a well-defined blurry spot or blind spot in the field of vision, general haziness in overall vision, and abrupt onset and rapid worsening of symptoms. Treatments include medications directed to stopping the growth of new blood vessels, such as bevacizumab (AVASTIN®), ranibizumab (LUCENTIS®), and aflibercept (EYLEA®); photodynamic therapy; photocoagulation; and low vision rehabilitation.

Agent: Any polypeptide, compound, small molecule, organic compound, salt, polynucleotide, or other molecule of interest. Agent can include a therapeutic agent, a diagnostic agent or a pharmaceutical agent. A therapeutic agent is a substance that demonstrates some therapeutic effect by restoring or maintaining health, such as by alleviating the symptoms associated with a disease or physiological disorder, or delaying (including preventing) progression or onset of a disease, such as, but not limited to, age-related retinal diseases.

Aliphatic: A hydrocarbon, or a radical thereof, having at least one carbon atom to 50 carbon atoms, such as one to 25 carbon atoms, or one to ten carbon atoms, and which includes alkanes (or alkyl), alkenes (or alkenyl), alkynes (or alkynyl), including cyclic versions thereof, and further including straight- and branched-chain arrangements, and all stereo and position isomers as well.

Alkenyl: An unsaturated monovalent hydrocarbon having at least two carbon atoms to 50 carbon atoms, such as two to 25 carbon atoms, or two to ten carbon atoms, and at least one carbon-carbon double bond, wherein the unsaturated monovalent hydrocarbon can be derived from removing one hydrogen atom from one carbon atom of a parent alkene. An alkenyl group can be branched, straight-chain, cyclic (such as cycloalkenyl), cis, or trans (such as E or Z).

Alkyl: A saturated monovalent hydrocarbon having at least one carbon atom to 50 carbon atoms, such as one to 25 carbon atoms, or one to ten carbon atoms, wherein the saturated monovalent hydrocarbon can be derived from removing one hydrogen atom from one carbon atom of a parent compound (such as alkane). An alkyl group can be branched, straight-chain, or cyclic (such as cycloalkyl).

Alkoxyl: A univalent radical R—O—, or anion R—O—, wherein R is an alkyl group.

Alkynyl: An unsaturated monovalent hydrocarbon having at least two carbon atoms to 50 carbon atoms, such as two to 25 carbon atoms, or two to ten carbon atoms and at least one carbon-carbon triple bond, wherein the unsaturated monovalent hydrocarbon can be derived from removing one hydrogen atom from one carbon atom of a parent alkyne. An alkynyl group can be branched, straight-chain, or cyclic (such as cycloalkynyl).

Amide: —NC(O)R, wherein R is hydrogen, aliphatic, aryl, heteroaliphatic, or heteroaryl.

Amine: —NR′R, wherein each of R and R′ independently are hydrogen, aliphatic, aryl, heteroaliphatic, or heteroaryl.

Analog: A compound with a molecular structure closely similar to that of another, such as an analog of the transition state of a substrate during catalysis (for example, a transition state analog of catalysis by PNPase).

Animal: Living multi-cellular vertebrate organisms, a category that includes, for example, mammals and birds. The term mammal includes both human and non-human mammals. Similarly, the term “subject” includes both human and veterinary subjects.

Aryl: An aromatic carbocyclic group comprising at least five carbon atoms to 15 carbon atoms, such as five to ten carbon atoms, having a single ring or multiple condensed rings, which condensed rings can or may not be aromatic provided that the point of attachment is through an atom of the aromatic carbocyclic group.

Carbonate: —OC(O)OR, wherein R is hydrogen, aliphatic, aryl, heteroaliphatic, or heteroaryl.

Carbonyl: C═O, wherein the carbon located at the 8 position of guanine or guanosine forms a double bond with an oxygen atom.

Carboxyl: —C(O)OR, wherein R is hydrogen, aliphatic, aryl, heteroaliphatic, or heteroaryl.

Cone-Rod Dystrophy: The first signs and symptoms of cone-rod dystrophy, which often occur in childhood, are usually decreased sharpness of vision (visual acuity) and increased sensitivity to light (photophobia). These features are typically followed by impaired color vision (dyschromatopsia), blind spots (scotomas) in the center of the visual field, and partial side (peripheral) vision loss. Over time, affected individuals develop night blindness and a worsening of their peripheral vision, which can limit independent mobility. The cone dystrophy is characterized by progressive dysfunction of the photopic system with preservation of scotopic function. Abnormal rod function may be part of the initial presentation, but rod involvement may be less severe or occur later than the cone dysfunction.

Control subject: A control subject is a subject that is used to provide a basis for comparison. As a comparison to subjects with or at risk for a particular condition (such as a subject that has an age-related retinal disease), control subjects may belong to a group of healthy subject who are studied to observe how their symptoms, traits, or behaviors compare to a group of subjects with or at risk for a particular condition.

Cytokines: A broad category of small proteins (approximately 5-20 kDa) that are important in cell signaling. Their release has an effect on the behavior of cells around them. Cytokines are involved in autocrine signaling, paracrine signaling and endocrine signaling as immunomodulating agents. Cytokines include chemokines, interferons, interleukins, lymphokines, and tumor necrosis factors but generally not hormones or growth factors. Cytokines are produced by a broad range of cells, including immune cells, such as macrophages, B lymphocytes, T lymphocytes, and mast cells, as well as endothelial cells, fibroblasts, and various stromal cells; a given cytokine may be produced by more than one type of cell. Cytokines are important in health and disease, specifically in host responses to infection, immune responses, inflammation, trauma, sepsis, cancer, and reproduction. They act through receptors and are especially important in the immune system; cytokines modulate the balance between humoral and cell-based immune responses, and they regulate the maturation, growth, and responsiveness of particular cell populations. Cytokines include interleukins, such as IL-1beta, and chemoattractants, such as monocyte chemoattractant protein-1 (MCP-1).

Diabetic retinopathy: Diabetic retinopathy is a diabetes complication in which the blood vessels of the retinal tissue is damaged, symptoms of which can range from absent or mild, such as at onset, to blindness. In some embodiments, diabetic retinopathy is diagnosed with a comprehensive dilated eye exam, for example, to identify abnormal blood vessels; swelling, blood or fatty deposits in the retina; growth of new blood vessels and scar tissue; bleeding in the clear, jelly-like substance that fills the center of the eye (vitreous); retinal detachment; and abnormalities in the optic nerve. Additional diagnostic examinations include vision, glaucoma, and cataract tests as well as a fluorescein angiography or optical coherence tomography, for example, to determine whether fluid has leaked into the retinal tissue. Treatment includes photocoagulation, focal laser treatment, panretinal photocoagulation, vitrectomy, and intravitreal administration of medications, such as vascular endothelial growth factor (VEGF) inhibitors.

Ester: —OC(O)R, wherein R is hydrogen, aliphatic, aryl, heteroaliphatic, or heteroaryl.

Glaucoma: Glaucoma is characterized by damage the optic nerve, which can be caused, for example, by an abnormally high pressure in the eye, and can cause of blindness, for example, in people over the age of 60, but can occur at any age. Symptoms include patchy blind spots in the side (peripheral) or central vision and tunnel vision in advanced stages, such as in acute angle-closure glaucoma, and severe headache, eye pain, nausea and vomiting, blurred vision, halos around lights, and eye redness, such as in acute angle-closure glaucoma. In some examples, glaucoma can be diagnosed by measuring intraocular pressure (tonometry), testing for optic nerve damage with dilated eye examination and imaging tests, examining for areas of vision loss (visual field test), measuring corneal thickness (pachymetry), and/or inspecting the drainage angle (gonioscopy). In some examples, treatments can include medication (such as delivered through eyedrops or orally), such as prostaglandins (for example, latanoprost (XALATAN®), travoprost (TRAVATAN Z®), tafluprost (ZIOPTAN®), bimatoprost (LUMIGAN®), and latanoprostene bunod (VYZULTA®)); beta blockers (such as timolol (BETIMOL®, ISTALOL®, and TIMOPTIC®) and betaxolol (BETOPTIC®)); alpha-adrenergic agonists (such as apraclonidine (IOPIDINE®) and brimonidine (ALPHAGAN P®, QOLIANA®)); carbonic anhydrase inhibitors (such as dorzolamide (Trusopt®) and brinzolamide (AZOPT®)); rho kinase inhibitors (such as netarsudil (RHOPRESSA®)); miotic or cholinergic agents (such as pilocarpine (ISOPTO CARPINE®)); laser therapy (such as a laser trabeculoplasty; filtering surgery (such as a trabeculectomy); drainage tubes; and minimally invasive glaucoma surgery (MIGS).

Halogen: bromo, fluoro, iodo, or chloro.

Heteroaliphatic: An aliphatic group comprising at least one heteroatom to 20 heteroatoms, such as one to 15 heteroatoms, or one to 5 heteroatoms, which can be selected from, but not limited to oxygen, nitrogen, sulfur, selenium, phosphorous, and oxidized forms thereof within the group.

Heteroalkyl/Heteroalkenyl/Heteroalkynyl: An alkyl, alkenyl, or alkynyl group (which can be branched, straight-chain, or cyclic) comprising at least one heteroatom to 20 heteroatoms, such as one to 15 heteroatoms, or one to 5 heteroatoms, which can be selected from, but not limited to oxygen, nitrogen, sulfur, selenium, phosphorous, and oxidized forms thereof within the group.

Heteroaryl: An aryl group comprising at least one heteroatom to six heteroatoms, such as one to four heteroatoms, which can be selected from, but not limited to oxygen, nitrogen, sulfur, selenium, phosphorous, and oxidized forms thereof within the ring. Such heteroaryl groups can have a single ring or multiple condensed rings, wherein the condensed rings may or may not be aromatic or contain a heteroatom, provided that the point of attachment is through an atom of the aromatic heteroaryl group.

Hydroxyl: —OH.

Hypertensive retinopathy: Hypertensive retinopathy can occur with acute blood pressure elevation, which can cause reversible vasoconstriction in retinal blood vessels and optic disk edema, which leads to exudative vascular changes with severe or prolonged hypertension; arteriole wall thickening; and arteriovenous nicking. Diagnosis can include a history of hypertension and a funduscopy. Treatment can include controlling hypertension, retinal laser therapy, or intravitreal injection of corticosteroids or antivascular endothelial growth factor drugs (such as ranibizumab, pegaptanib, or bevacizumab).

Inflammation: Inflammation is a localized protective response elicited by injury to tissue that serves to sequester the inflammatory agent. Inflammation is orchestrated by a complex biological response of vascular tissues to harmful stimuli, such as pathogens, damaged cells, or irritants. It is a protective attempt by the organism to remove the injurious stimuli as well as initiate the healing process for the tissue. An inflammatory response is characterized by an accumulation of white blood cells, either systemically or locally at the site of inflammation. The inflammatory response may be measured by many methods, including, but not limited to measuring the number of white blood cells, the number of polymorphonuclear neutrophils (PMN), a measure of the degree of PMN activation, such as luminol enhanced-chemiluminescence, or a measure of the amount of cytokines present. C-reactive protein is a marker of a systemic inflammatory response.

Interleukin 1-beta (IL-1β): Also known as IL1B, IL1-beta, leukocytic pyrogen, leukocytic endogenous mediator, mononuclear cell factor, and lymphocyte activating factor (e.g., OMIM 147720), IL-1β is a cytokine produced by activated macrophages and is an important mediator of the inflammatory response. IL-1β is involved in a variety of cellular activities, including cell proliferation, differentiation, and apoptosis. Increased production and/or activity of IL-1β causes multiple autoinflammatory syndromes and has been linked to susceptibility to cancer and tuberculosis. Exemplary protein and nucleotide sequences for IL-1β are available at GENBANK® (e.g., Accession Nos. NP_000567.1 and NM_000576.3, respectively, incorporated by reference herein as available on Mar. 2, 2020).

Intraocular administration: Administering agents locally, directly into the eye, for example by delivery into the vitreous or anterior chamber, or sub-retinally. Indirect intraocular delivery (for example, by diffusion through the cornea) is not direct administration into the eye.

Intravitreal administration: Administering agents into the vitreous cavity. The vitreous cavity is the space that occupies most of the volume of the core of the eye with the lens and its suspension system (the zonules) as its anterior border and the retina and its coating as the peripheral border. Intravitreal administration can be accomplished by injection, pumping, or by implants.

LCA: A rare inherited eye disease that appears at birth or in the early stages of life (infancy or early childhood) and primarily affects the retina. The presentation can vary because is it associated with multiple genes. However, it is characterized by characterized by nystagmus, photophobia, sluggish or absent pupillary response, and severe vision loss or blindness. The common modes of inheritance are autosomal recessive and autosomal dominant.

The pupils, which usually expand and contract in response to the amount of light entering the eye, do not react normally to light. Instead, they expand and contract more slowly than normal, or they may not respond to light at all. Additionally, the clear front covering of the eye (the cornea) may be cone-shaped and abnormally thin, a condition known as keratoconus. A specific behavior referred to as Franceschetti's oculo-digital sign is characteristic of LCA. This sign consists of poking, pressing, and rubbing the eyes with a knuckle or finger.

Nitro: —NO₂.

Nitroso: —NO.

Pharmaceutically acceptable carrier: As used herein, the term “pharmaceutically acceptable carrier” includes any and all solvents, dispersion media, coatings, surfactants, antioxidants, preservatives (such as antibacterial agents, antifungal agents), isotonic agents, absorption delaying agents, salts, preservatives, drugs, drug stabilizers, binders, excipients, disintegration agents, lubricants, sweetening agents, flavoring agents, dyes, such like materials and combinations thereof, as would be known to one of ordinary skill in the art (see, such as Remington's Pharmaceutical Sciences, 1289-1329, 1990, incorporated herein by reference). Except insofar as any conventional carrier is incompatible with the active ingredient, its use in the therapeutic or pharmaceutical compositions is contemplated.

PNPase: Purine nucleoside phosphorylase, a glycosyltransferase, is an enzyme that catalyzes a chemical reaction between purine nucleoside (such as the PNPase purine nucleoside substrates inosine and guanosine) and phosphate. PNPase inhibitors inhibit the catalytic action of a PNPase. Non-limiting examples of PNPase inhibitors include 8-substituted guanine, guanosine, inosine, and hypoxanthine compounds (for example, 8-aminoguanine, 8-aminoguanosine, 8-aminoinosine, and 8-aminohypoxanthine); and PNPase transition state analogs, such as forodesine, or a forodesine derivative, for example, DADMe-immucillin-H, DATMe-immucillin-H, or SerMe-immucillin-H, or as described in U.S. Pat. Nos. 4,985,433; 4,985,434, 5,008,265; 5,008,270; 5,565,463, and 5,721,240 as well as US pat. pub. no. 2018/0258091A1, all of which are incorporated herein by reference in their entireties).

Retina: The retina is the innermost, light-sensitive layer of tissue of the eye of most vertebrates. The optics of the eye create a focused two-dimensional image on the retina, which translates that image into electrical neural impulses to the brain. The neural retina includes several layers of neurons interconnected by synapses, supported by an outer layer of pigmented epithelial cells. Photoreceptor cells are included: rods and cones. Rods function in dim light and provide black-and-white vision; cones function in well-lit conditions and are responsible for color perception and high-acuity vision. Photosensitive ganglion cell are also included, which play a role in circadian rhythms and reflexive responses, such as the pupillary light reflex.

Retinal detachment: Retinal detachment is a condition in which the retina is pulled away from its normal position with symptoms that include a sudden increase in the number of specks floating in your vision (floaters), flashes of light in one eye or both eyes, a “curtain” or shadow over your field of vision, and, without immediate treatment, permanent vision loss.

Retinal diseases and disorders: Retinal diseases include disease in which the function or structure of the retina is damaged or decreased. Retinal degenerative diseases are included, in which the retinal structure or function changes for the worse over time. Retinal vascular diseases are included, such as retinal diseases in which the structure or function of the blood vessels in the eye are affected, for example, hypertensive retinopathy, retinal RVO, CRAO, BRAO, and diabetic retinopathy. Examples include LCA, RP, cone rod dystrophy, AMD, diabetic retinopathy, and retinal detachment.

Retinal occlusion: Retinal occlusion can occur when one of the retinal veins or arteries is blocked. Blockage of retinal veins is referred to as RVO, or central or branch RVO if the blockage is a main or smaller retinal vein, respectively. Blockage of retinal arteries is referred to as RAO, or CRAO or branch BRAO if the blockage is a main or smaller retinal vein, respectively. Ocular coherence tomography (OCT) and fluorescein angiography can be used to diagnose retinal occlusions, and treatment can include intravitreal injections of medication, such as anti-VEGF (for example, Lucentis, Avastin, or Eylea) or steroids; laser therapy; or surgery (such as vitrectomy surgery).

RP: RP is an inherited, degenerative eye disease that causes severe vision impairment due to the progressive degeneration of the rod photoreceptor cells in the retina. This form of retinal dystrophy manifests initial symptoms independent of age. The initial retinal degenerative symptoms of RP are characterized by decreased night vision (nyctalopia) and the loss of the mid-peripheral visual field. The rod photoreceptor cells, which are responsible for low-light vision and are orientated in the retinal periphery, are the retinal processes affected first during non-syndromic forms of this disease. Visual decline progresses relatively quickly to the far peripheral field, eventually extending into the central visual field as tunnel vision increases. Visual acuity and color vision can become compromised due to accompanying abnormalities in the cone photoreceptor cells, which are responsible for color vision, visual acuity, and sight in the central visual field. The progression of disease symptoms occurs in a symmetrical manner, with both the left and right eyes experiencing symptoms at a similar rate.

Subject: As used herein, the term “subject” refers to a mammal and includes, without limitation, humans and veterinary subjects, including domestic animals (such as dogs or cats), farm animals (such as cows, horses, or pigs), and laboratory animals (such as mice, rats, hamsters, guinea pigs, pigs, rabbits, dogs, or monkeys). In humans, and “older” subject is a subject that is more than 50 years of age.

Transition state analog: A chemical compound with a chemical structure that resembles the transition state of a substrate molecule in an enzyme-catalyzed chemical reaction. A ‘PNPase transition state analog’ is a compound that resembles the transition state of the reaction (such as catalysis of inosine to hypoxanthine or catalysis of guanosine to guanine) catalyzed by PNPase. These compounds act as an inhibitor of the PNPase by blocking its active site.

Therapeutically effective amount: The term “therapeutically effective amount” refers to the amount of an active ingredient (such as, but not limited to, a PNPase transition state analog, 8-substituted guanine, 8-substituted guanosine, 8-substituted guanosine, and 8-substituted inosine) that is sufficient to effect treatment when administered to a mammal that has an age-related retinal disease. The therapeutically effective amount will vary depending upon the subject and disease condition being treated, the weight and age of the subject, the severity of the disease condition, the manner of administration and the like, which can readily be determined by a prescribing physician.

Treating or inhibiting a disease: Inhibiting the full development of a disease or condition, for example, in a subject who has, or is at risk for, a disease such as an age-related retinal disease. “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, an improvement in the overall health or well-being of the subject, or by other parameters well known in the art that are specific to the particular disease. “Prophylaxis” is a treatment administered to a subject who does not exhibit signs of a disease or exhibits only early signs for the purpose of decreasing the risk of developing pathology.

Vasculature: The vasculature can include the circulatory system (such as an arrangement of blood vessels) or a portion thereof, such as a supply of vessels to a specific region.

### II. Overview

Methods for using a PNPase inhibitor or a PNPase purine nucleoside substrate are disclosed herein. Many diseases in which 8-AG, 8-aminoguanosine, 8-hydroxyguanine, 8-hydroxyguanosine, 8-nitroguanine, 8-aminoinosine, 8-aminohypoxanthine, forodesine, and other PNPase inhibitors or substrates, such as other 8-substituted guanine, guanosine, inosine, and hypoxanthine compounds as well as other PNPase transition state analogs are useful are enriched in the elderly population. In some embodiments, the PNPase inhibitor is a guanine with a substituent at the 8-position, a guanosine with a substituent at the 8-position, an inosine with a substituent at the 8-position, a hypoxanthine with a substituent at the 8-position, a PNPase transition state analog, or a pharmaceutically acceptable salt thereof. In some non-limiting examples, the substituent is amine, hydroxyl, nitro, nitroso, alkoxy, carbonyl, halogen, carboxyl, ester, carbonate, amide, or haloaliphatic. In particular non-limiting examples, the substituent is amine. In some examples, the guanine includes a substituent at the 8-position is 8-AG. In some examples, the PNPase transition state analog is 7-[(2S,3S,4R,5R)-3,4-dihydroxy-5-(hydroxymethyl)pyrrolidin-2-yl]-3H,4H,5H-pyrrolo[3,2-d]pyrimidin-4-one; 7-(((3R,4R)-3-hydroxy-4-(hydroxymethyl)pyrrolidin-1-yl)methyl)-3H-pyrrolo[3,2-d]pyrimidin-4(5H)-one; 7-(((2R,3S)-1,3,4-trihydroxybutan-2-ylamino)methyl)-3H-pyrrolo[3,2-d]pyrimidin-4(5H)-one; 7-((1,3-dihydroxypropan-2-ylamino)methyl)-3H-pyrrolo[3,2-d]pyrimidin-4(5H)-one; or a pharmaceutically acceptable salt thereof (such as a chloride salt). In some examples, the PNPase transition state analog or pharmaceutically acceptable salt thereof is administered intravenously or into the eye of the subject. In some examples, the PNPase inhibitor is a guanine with a substituent at the 8-position or a guanosine with a substituent at the 8-position, and is administered orally, intravenously, or into the eye (such as on the conjunctiva) of the subject.

In some examples, the subject has cataracts, ganglion cell degeneration, glaucoma, a retinal disease, conjunctivitis, a corneal disease, eyelid problems, or temporal arteritis. In some examples, the subject has a retinal disease, such as a retinal vascular disease, LCA, RP, cone rod dystrophy, AMD, or retinal detachment. In some examples, the subject has a retinal vascular disease, such as hypertensive retinopathy, RVO, CRAO, BRAO, or diabetic retinopathy. In some examples, the subject has AMD.

In some examples, the administering includes delivering the PNPase inhibitor or PNPase purine nucleoside substrate into the eye of the subject. In some examples, the administering includes repeated delivering the PNPase inhibitor or PNPase purine nucleoside substrate into the eye of the subject. In some examples, the administering to the subject a therapeutically effective amount of a purine nucleoside phosphorylase (PNPase) inhibitor or a PNPase purine nucleoside substrate produces a decrease in at least one inflammatory cytokine (such as interleukin 1 beta (IL-1beta) or monocyte chemoattractant protein-1 (MCP-1)).

In some examples, the subject is a veterinary subject or a human subject, such as a human subject at least 50 years old.

### III. Therapeutic Compounds, Pharmaceutical Compositions, and Methods of Administration

It is disclosed herein that a PNPase inhibitor or a PNPase purine nucleoside substrate (for example, PNPase purine nucleoside substrates that can act as both a substrate or a PNPase inhibitor) can be used therapeutically for the treatment of age-related retinal disease. The disclosed methods reduce inflammation and improve vasculature in a retina of a subject.

Examples of (PNPase) inhibitor or a PNPase purine nucleoside substrates that can be used therapeutically for the treatment of age-related retinal disease include guanine; guanosine; inosine; hypoxanthine; amiloride; an 8-substituted guanine (such as 8-AG, 8-hydroxyguanine, or 8-nitroguanine), guanosine (such as 8-aminoguanosine or 8-hydroxyguanosine), inosine (such as 8-aminoinosine), or hypoxanthine (such as 8-aminohypoxanthine); forodesine or a derivative thereof (for example, DADMe-Immucillin-H, DATMe-Immucillin-H, or SerMe-Immucillin-H, or such as described in U.S. Pat. Nos. 4,985,433; 4,985,434, 5,008,265; 5,008,270; 5,565,463, and 5,721,240 as well as US pat. pub. no. 2018/0258091A1, all of which are incorporated herein by reference in their entireties), or a pharmaceutically acceptable salt thereof (such as a chloride salt). The 8-substituted guanine, guanosine, inosine, and hypoxanthine compounds are referred to as Formula 1 (guanine with a substituent at the 8 position), Formula 2 (guanosine with a substituent at the 8 position), Formula 3 (hypoxanthine with a substituent at the 8 position), and Formula 4 (inosine with a substituent at the 8 position), respectively. The general chemical structures of these compounds are shown below and are further defined in the Terms section.

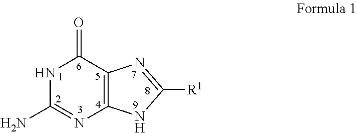

With reference to Formula 1, R¹ is selected from amine (—NR′R), hydroxyl (—OH), nitro (—NO₂), nitroso (—NO), alkoxy, carbonyl (C═O), halogen, carboxyl, ester, carbonate, amide, haloaliphatic, or hydrogen.

In some embodiments of Formula 1, R¹ is amine (—NR′R), and each of R and R′ independently are hydrogen, aliphatic, aryl, heteroaliphatic, or heteroaryl.

In some embodiments of Formula 1, R¹ is carbonyl (C═O), and the carbon located at the 8 position of Formula 1 forms a double bond with an oxygen atom.

In some embodiments of Formula 1, R¹ is halogen, and the halogen is bromo, fluoro, iodo, or chloro.

In some embodiments of Formula 1, R¹ is carboxyl (—C(O)OR), and R is hydrogen, aliphatic, aryl, heteroaliphatic, or heteroaryl.

In some embodiments of Formula 1, R¹ is ester (—OC(O)R), and R is hydrogen, aliphatic, aryl, heteroaliphatic, or heteroaryl.

In some embodiments of Formula 1, R¹ is carbonate (—OC(O)OR), and R is hydrogen, aliphatic, aryl, heteroaliphatic, or heteroaryl.

In some embodiments of Formula 1, R¹ is amide (—NC(O)R), and R is hydrogen, aliphatic, aryl, heteroaliphatic, or heteroaryl.

In some embodiments of Formula 1, R¹ is haloaliphatic (—CH₂X, —CHX₂, or —CX₃), and each X independently is halogen (Cl, Br, F, or I).

Any of the compound embodiments of Formula 1 can be included in a pharmaceutical composition and used in the methods disclosed herein.

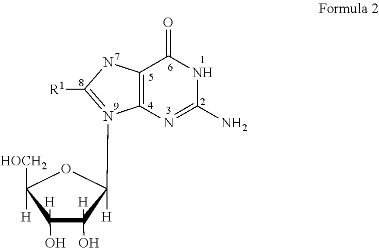

With reference to Formula 1, R¹ is selected from amine (—NR′R), hydroxyl (—OH), nitro (—NO₂), nitroso (—NO), alkoxy, carbonyl (C═O), halogen, carboxyl, ester, carbonate, amide, haloaliphatic, or hydrogen.

In some embodiments of Formula 2, R¹ is amine (—NR′R), and each of R and R′ independently are hydrogen, aliphatic, aryl, heteroaliphatic, or heteroaryl.

In some embodiments of Formula 2, R¹ is carbonyl (C═O), and the carbon located at the 8 position of Formula 1 forms a double bond with an oxygen atom.

In some embodiments of Formula 2, R¹ is halogen, and the halogen is bromo, fluoro, iodo, or chloro.

In some embodiments of Formula 2, R¹ is carboxyl (—C(O)OR), and R is hydrogen, aliphatic, aryl, heteroaliphatic, or heteroaryl.

In some embodiments of Formula 2, R¹ is ester (—OC(O)R), and R is hydrogen, aliphatic, aryl, heteroaliphatic, or heteroaryl.

In some embodiments of Formula 2, R¹ is carbonate (—OC(O)OR), and R is hydrogen, aliphatic, aryl, heteroaliphatic, or heteroaryl.

In some embodiments of Formula 2, R¹ is amide (—NC(O)R), and R is hydrogen, aliphatic, aryl, heteroaliphatic, r heteroaryl.

In some embodiments of Formula 2, R¹ is haloaliphatic (—CH₂X, —CHX₂, or —CX₃), and each X independently is halogen (Cl, Br, F, or I).

Any of the compound embodiments of Formula 2 can be included in a pharmaceutical composition and used in the methods disclosed herein.

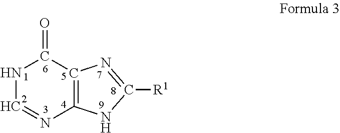

With reference to Formula 3, R¹ is selected from amine (—NR′R), hydroxyl (—OH), nitro (—NO₂), nitroso (—NO), alkoxy, carbonyl (C═O), halogen, carboxyl, ester, carbonate, amide, haloaliphatic, or hydrogen.

In some embodiments of Formula 3, R¹ is amine (—NR′R), and each of R and R′ independently are hydrogen, aliphatic, aryl, heteroaliphatic, or heteroaryl.

In some embodiments of Formula 3, R¹ is carbonyl (C═O), and the carbon located at the 8 position of Formula 1 forms a double bond with an oxygen atom.

In some embodiments of Formula 3, R¹ is halogen, and the halogen is bromo, fluoro, iodo, or chloro.

In some embodiments of Formula 3, R¹ is carboxyl (—C(O)OR), and R is hydrogen, aliphatic, aryl, heteroaliphatic, or heteroaryl.

In some embodiments of Formula 3, R¹ is ester (—OC(O)R), and R is hydrogen, aliphatic, aryl, heteroaliphatic, or heteroaryl.

In some embodiments of Formula 3, R¹ is carbonate (—OC(O)OR), and R is hydrogen, aliphatic, aryl, heteroaliphatic, or heteroaryl.

In some embodiments of Formula 3, R¹ is amide (—NC(O)R), and R is hydrogen, aliphatic, aryl, heteroaliphatic, or heteroaryl.

In some embodiments of Formula 3, R¹ is haloaliphatic (—CH₂X, —CHX₂, or —CX₃), and each X independently is halogen (Cl, Br, F, or I).

Any of the compound embodiments of Formula 3 can be included in a pharmaceutical composition and used in the methods disclosed herein.

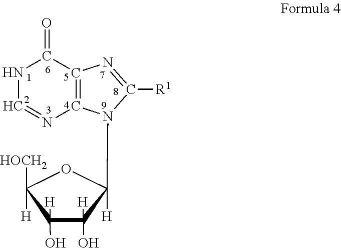

With reference to Formula 4, R¹ is selected from amine (—NR′R), hydroxyl (—OH), nitro (—NO₂), nitroso (—NO), alkoxy, carbonyl (C═O), halogen, carboxyl, ester, carbonate, amide, haloaliphatic, or hydrogen.

In some embodiments of Formula 4, R¹ is amine (—NR′R), and each of R and R′ independently are hydrogen, aliphatic, aryl, heteroaliphatic, or heteroaryl.

In some embodiments of Formula 4, R¹ is carbonyl (C═O), and the carbon located at the 8 position of Formula 4 forms a double bond with an oxygen atom.

In some embodiments of Formula 4, R¹ is halogen, and the halogen is bromo, fluoro, iodo, or chloro.

In some embodiments of Formula 4, R¹ is carboxyl (—C(O)OR), and R is hydrogen, aliphatic, aryl, heteroaliphatic, or heteroaryl.

In some embodiments of Formula 4, R¹ is ester (—OC(O)R), and R is hydrogen, aliphatic, aryl, heteroaliphatic, or heteroaryl.

In some embodiments of Formula 4, R¹ is carbonate (—OC(O)OR), and R is hydrogen, aliphatic, aryl, heteroaliphatic, or heteroaryl.

In some embodiments of Formula 4, R¹ is amide (—NC(O)R), and R is hydrogen, aliphatic, aryl, heteroaliphatic, or heteroaryl.

In some embodiments of Formula 4, R¹ is haloaliphatic (—CH₂X, —CHX₂, or —CX₃), and each X independently is halogen (Cl, Br, F, or I).

Any of the compound embodiments of Formula 4 can be included in a pharmaceutical composition and used in the methods disclosed herein.

Specific compounds of use in the methods disclosed herein include those shown in Table 2.

TABLE 2
Chemical structures for compounds of use in the methods disclosed herein

| Name | Structure |
|---|---|
| Guanine | 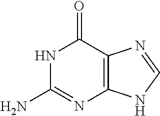 [SMILES: Nc1nc2[nH]cnc2c(=O)[nH]1] |
| 8-AG | 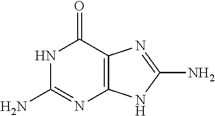 [SMILES: Nc1nc2[nH]c(N)nc2c(=O)[nH]1] |
| 8-Hydroxyguanine | 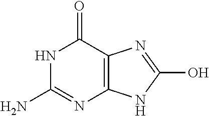 [SMILES: Nc1nc2[nH]c(O)nc2c(=O)[nH]1] |
| 8-Nitroguanine | 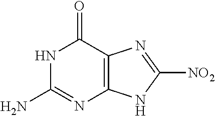 [SMILES: Nc1nc2[nH]c([N+](=O)[O-])nc2c(=O)[nH]1] |
| Inosine | 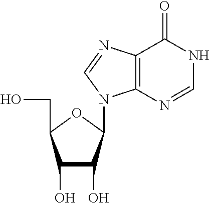 |
| 8-aminoinosine | 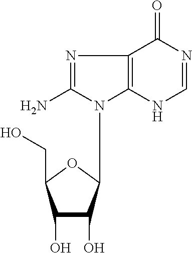 |
| Guanosine | 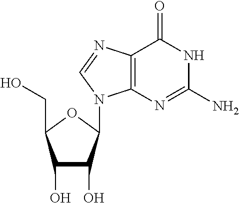 |
| 8-Aminoguanosine | 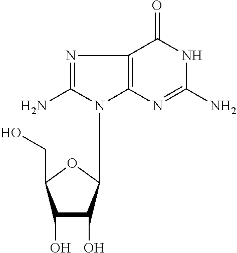 |
| 8-Hydroxyguanosine | 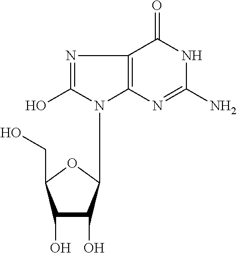 |
| Amiloride | 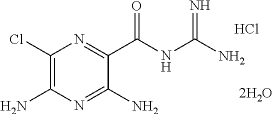 [SMILES: N=C(N)NC(=O)c1nc(Cl)c(N)nc1N ; Cl] |
| Hypoxanthine | 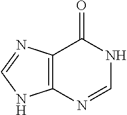 |
| 8-aminohypoxanthine | 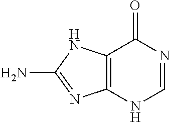 [SMILES: Nc1nc2[nH]cnc(=O)c2[nH]1] |
| Name(s) | Structure |
| Forodesine, Immucillin-H, and 7-[(2S,3S,4R,5R)-3,4- dihydroxy-5-(hydroxymethyl)pyrrolidin-2-yl]-3H,4H,5H- pyrrolo[3,2-d]pyrimidin-4-one | 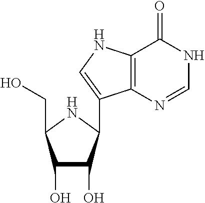 |
| DADMe-Immucillin-H, Ulodesine, and 7-(((3R,4R)-3- hydroxy-4-(hydroxymethyl)pyrrolidin-1-yl)methyl)-3H- pyrrolo[3,2-d]pyrimidin-4(5H)-one | 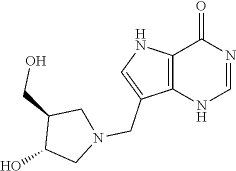 [SMILES: O=c1nc[nH]c2c(CN3C[C@H](CO)[C@@H](O)C3)c[nH]c12] |
| DATMe-Immucillin-H and 7-(((2R,3S)-1,3,4- trihydroxybutan-2-ylamino)methyl)-3H-pyrrolo[3,2- d]pyrimidin-4(5H)-one | 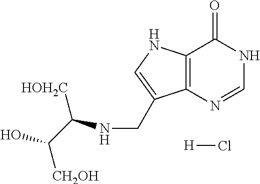 |
| SerMe-Immucillin-H and 7-((1,3-dihydroxypropan-2- ylamino)methyl)-3H-pyrrolo[3,2-d]pyrimidin-4(5H)-one | 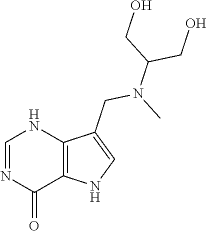 [SMILES: CN(Cc1c[nH]c2c(=O)nc[nH]c12)C(CO)CO] |

Compounds of use in the disclosed method include PNPase transition state analogs. As disclosed in U.S. Pat. No. 5,721,240, incorporated herein by reference in its entirety, 9-arylmethyl-substituted purines (including guanines) have been reported as PNP inhibitors in U.S. Pat. No. 4,772,606. PNPase inhibitory data cited in Drugs of the Future 13, 654 (1988) and Agents and Actions 21, 253 (1987) indicate that the 9-arylmethyl (Ar) substituted guanine derivatives of the formula:

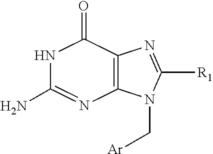

wherein R₈ represents hydrogen are markedly less potent PNP inhibitors than the corresponding compounds wherein R₈ represents amino (8-AGs).

This U.S. patent disclosed inhibitors of the formula:

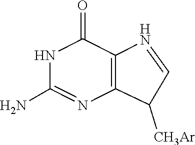

wherein CH₂Ar represents:

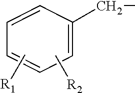

which R₁ represents hydrogen, halogen, C₁-C₃-alkyl, C₁-C₃-alkoxy, benzyloxy, hydroxy or trifluoromethyl; and R represents hydrogen, halogen, C₁-C₃-alkyl, C₁-C₃-alkoxy, benzyloxy, hydroxy or trifluoromethyl; provided that R₂ represents hydrogen or C₁-C₃-alkyl if R₁ represents trifluoromethyl, or that R₁ represents hydrogen or C₁-C₃-alkyl if R₂ represents trifluoromethyl; or
wherein CH₂Ar represents:

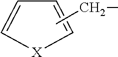

in which X represents sulfur or oxygen and in which attachment to the thiophene or furan ring is at the 2- or 3-position; and tautomers thereof.

This U.S. Patent also discloses compounds specified by formulas II, III, and IV, which can also be used in the presently disclosed methods. Pharmaceutically acceptable salts and hydrates are also disclosed. In one embodiment, and pharmaceutically acceptable salt is a chloride salt. However, other salts can be utilized, such as alkali metal salts; esters such as acetate, butyrate, octinoate, palmitate, chlorobenzoates, benzoates, C₁-C₆ benzoates, succinates, and mesylate; salts of such esters; and nitrile oxides.

PCT Publication No. WO99/19338, incorporated herein by reference, discloses a compound genus as a new class of inhibitors of nucleoside metabolism, including forodesine, all of which can be used in the presently disclosed methods. PCT Publication No. WO 2016/110527, also incorporated herein by reference discloses methods for synthesis of forodesine. Forodesine, also known as immucillin-H and 7-[(2S,3S,4R,5R)-3,4-dihydroxy-5-(hydroxymethyl)-2-pyrrolidinyl]-1,5-dihydropyrrolo[2,3-e]pyrimidin-4-one, is an inhibitor of purine nucleoside phosphorylase.

In specific, non-limiting examples, the transition state analog can be forodesine (also known as immucillin-H and 7-[(2S,3S,4R,5R)-3,4-dihydroxy-5-(hydroxymethyl)pyrrolidin-2-yl]-3H,4H,5H-pyrrolo[3,2-d]pyrimidin-4-one) as well as derivatives thereof, such as DADMe-immucillin-H (also known as ulodesine and 7-(((3R,4R)-3-hydroxy-4-(hydroxymethyl)pyrrolidin-1-yl)methyl)-3H-pyrrolo[3,2-d]pyrimidin-4(5H)-one), DATMe-immucillin-H (also known as 7-(((2R,3S)-1,3,4-trihydroxybutan-2-ylamino)methyl)-3H-pyrrolo[3,2-d]pyrimidin-4(5H)-one), SerMe-immucillin-H (also known as 7-((1,3-dihydroxypropan-2-ylamino)methyl)-3H-pyrrolo[3,2-d]pyrimidin-4(5H)-one). Compounds of use are disclosed in U.S. Pat. Nos. 4,985,433; 4,985,434, 5,008,265; 5,008,270; 5,565,463, and 5,721,240 as well as U.S. Published Application No. 2018/0258091A1, all of which are incorporated herein by reference in their entireties. Pharmaceutically acceptable salts of these compounds are also of use, such as, but not limited to, chloride salts. Other salts can be utilized, such as alkali metal salts; esters such as acetate, butyrate, octinoate, palmitate, chlorobenzoates, benzoates, C₁-C₆ benzoates, succinates, and mesylate; salts of such esters; and nitrile oxides.

Any of these compounds can be included in pharmaceutical compositions and used in the methods disclosed herein. These pharmaceutic compositions can be formulated for systemic or local delivery.

Pharmaceutical compositions that include a PNPase inhibitor, such as, but not limited to, 8-substituted guanine, 8-substituted guanosine, 8-substituted inosine, or 8-substituted hypoxanthine (for example, 8-AG, 8-aminoguanosine, 8-hydroxyguanine, 8-hydroxyguanosine, 8-nitroguanine, 8-aminoinosine, or 8-aminohypoxanthine); amiloride; a PNPase transition state analog, such as forodesine or a forodesine derivative, for example, DADMe-immucillin-H, DATMe-immucillin-H, or SerMe-immucillin-H; a PNPase purine nucleoside substrate (such as guanosine and inosine, which can also act as a PNPase inhibitor); or a pharmaceutically acceptable salt thereof (such as a chloride salt, for example, a PNPase transition state analog chloride salt) can be formulated with an appropriate pharmaceutically acceptable carrier.

The dosage form of the pharmaceutical composition will be determined by the mode of administration chosen. For instance, in addition to injectable fluids, topical, inhalation, oral, infusion, and suppository formulations can be employed. Inhalation preparations can be liquid (such as solutions or suspensions) and include mists, sprays, and the like. Oral formulations can be liquid (such as syrups, solutions, or suspensions) or solid (such as powders, pills, tablets, or capsules). Suppository preparations can also be solid, gel, or in a suspension form. Infusion preparations, administered by catheter, are generally administered as liquids. For solid compositions, conventional non-toxic solid carriers can include pharmaceutical grades of mannitol, lactose, cellulose, starch, or magnesium stearate. Actual methods of preparing such dosage forms are known, or will be apparent, to those skilled in the art.

The amount of PNPase inhibitor or PNPase purine nucleoside substrate administered will be dependent on the subject being treated, the severity of the affliction, and the manner of administration and is best left to the judgment of the prescribing clinician. Within these bounds, the formulation to be administered will contain a quantity of the active component(s) in amounts effective to achieve the desired effect in the subject being treated. A therapeutically effective amount of PNPase inhibitor or PNPase purine nucleoside substrate can be the amount of PNPase inhibitor or PNPase purine nucleoside substrate that is necessary to treat or lower the risk of a subject for a particular disease condition (see below).

The pharmaceutical compositions that include PNPase inhibitor (such as 8-substituted guanine, 8-substituted guanosine, 8-substituted inosine, 8-substituted hypoxanthine, amiloride, transition state analogs, or pharmaceutically acceptable salts thereof, for example, a transition state analog chloride salt) or PNPase purine nucleoside substrate (such as guanosine and inosine, which can also act as a PNPase inhibitor) can be formulated in unit dosage form, suitable for individual administration of precise dosages. A variety of dosages and dosing regimens are possible (for example, Kilpatrick et al., International Immunopharmacology, 3:541-548, 2003; Gandhi et al., Blood, 106(13):4253-4260, 2005, both of which are incorporated herein by reference in their entireties). In one specific, non-limiting example, a unit dosage (such as intravenous dosage) can contain about 1-50 μmoles/kg, such as about 1-5, 5-10, 10-20, 20-30, 30-40, or 40-50 μmoles/kg or about 33.5 μmoles/kg of a PNPase inhibitor or a PNPase purine nucleoside substrate. In other examples, a therapeutically effective amount of a PNPase inhibitor or a PNPase purine nucleoside substrate (such as oral dosage) is about 0.1-50 mg/kg, such as about 0.1-1, 1-5, 5-10, 5-20, 10-20, 20-30, 30-40, or 40-50 mg/kg or at least about 5, 10, 20, or 30 mg/kg (such as about 5-20 mg/kg/day). In further examples, a therapeutically effective amount of a PNPase inhibitor or a PNPase purine nucleoside substrate is about 0.1-50 mg/kg, such as about 0.1-1, 1-5, 5-10, 10-20, 20-30, 30-40, or 40-50 mg/kg or at least about 0.9, 4.4, or 8.8 mg/kg. In additional examples, a therapeutically effective amount of a PNPase inhibitor or a PNPase purine nucleoside substrate is about 1-10,000 μM, such as about 1-5, 1-10, 1-100, 1-500, 100-500, 10-1,000, or 100-10,000 μM or at least about 1, 10, 25, 50, 100, 500, 750, 1,000, 5,000, or 10,000 μM, such as about 10-1,000 μM. In specific non-limiting examples, 10-1,000 μM PNPase inhibitor or a PNPase purine nucleoside substrate can be used, such as for direct injection into the eye or for topical administration.

Pharmaceutically acceptable salts and hydrates are also disclosed. In one embodiment, and pharmaceutically acceptable salt is a chloride salt. However, other salts can be utilized, such as alkali metal salts; esters such as acetate, butyrate, octinoate, palmitate, chlorobenzoates, benzoates, C₁-C₆ benzoates, succinates, and mesylate; salts of such esters; and nitrile oxides.

A variety of administration regimens are possible (for example, Kilpatrick et al., International Immunopharmacology, 3:541-548, 2003; Gandhi et al., Blood, 106(13):4253-4260, 2005, both of which are incorporated herein by reference in their entireties). Administration with a therapeutically effective amount can be a single administration or multiple administrations. Administration can involve daily or multi-daily or less than daily (such as weekly, monthly, etc.) doses over a period of a few days to weeks or months, or even years. In a particular non-limiting example, administration involves once daily dose or twice daily dose. The particular mode/manner of administration and the dosage regimen will be selected by the attending clinician, taking into account the particulars of the case (such as the subject, the disease, the disease state/severity involved, the particular administration, and whether the treatment is prophylactic). In specific, non-limiting examples, administration can be oral, by direct administration via ocular delivery, or by intravenous delivery.

The dosage form of the pharmaceutical composition will be determined by the mode of administration chosen, which can be systemic or localized (such as to the eye). The pharmaceutically acceptable carriers and excipients useful in this disclosure are conventional (see, for example, Remington: The Science and Practice of Pharmacy, The University of the Sciences in Philadelphia, Editor, Lippincott, Williams, & Wilkins, Philadelphia, Pa., 21st Edition (2005)). For instance, parenteral formulations usually comprise injectable fluids that are pharmaceutically and physiologically acceptable fluid vehicles, such as water, physiological saline, other balanced salt solutions, aqueous dextrose, glycerol, or the like. In addition to injectable fluids, inhalational, and oral formulations can be employed. For solid compositions (such as powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, pH buffering agents, or the like, for example, sodium acetate or sorbitan monolaurate. Excipients that can be included are, for instance, proteins, such as human serum albumin or plasma preparations. Actual methods of preparing such dosage forms are known, or will be apparent, to those skilled in the art.

The compositions of this disclosure that include PNPase inhibitor (such as 8-substituted guanine, 8-substituted guanosine, 8-substituted inosine, 8-substituted hypoxanthine, amiloride, forodesine, or a forodesine derivative, for example, 8-AG, 8-aminoguanosine, 8-hydroxyguanine, 8-hydroxyguanosine, 8-nitroguanine, 8-aminoinosine, 8-aminohypoxanthine, DADMe-immucillin-H, DATMe-immucillin-H, or SerMe-immucillin-H) or PNPase purine nucleoside substrate (such as guanosine and inosine, which can also act as a PNPase inhibitor) can be administered to humans or other animals by any means, including systemically or locally.

In some examples, site-specific administration of the composition can be used. For example, PNPase inhibitor, PNPase purine nucleoside substrate, and/or PNPase transition state analog or pharmaceutically acceptable salt thereof (such as chloride salt) may be administered locally, and in some examples, components administered systemically may be modified or formulated to target the components to the eye. Local modes of administration include, by way of example, intraocular, intraorbital, subconjuctival, intravitreal, subretinal or transscleral routes. In an embodiment, significantly smaller amounts of the components (compared with systemic approaches) may exert an effect when administered locally (for example, intravitreally) compared to when administered systemically (for example, intravenously). Local modes of administration can reduce or eliminate the incidence of potential side effects. In one embodiment, components described herein are delivered subretinally, e.g., by subretinal injection. Subretinal injections may be made directly into the macular, e.g., submacular injection. In some examples, administration can be directly on the conjunctiva. Exemplary methods include intraocular injection (e.g., retrobulbar, subretinal, submacular, intravitreal and intrachoridal), iontophoresis, eye drops, and intraocular implantation (e.g., intravitreal, sub-Tenons and sub-conjunctival).

In some examples, the compositions disclosed herein can be administered systemically, such as orally or parenterally, for example, intravenously, intramuscularly, intraperitoneally (i.p.), intranasally, intradermally, intrathecally, subcutaneously, via catheter, via inhalation, or via suppository. In one non-limiting example, the composition is administered orally. For oral administration, the pharmaceutical compositions can take the form of, for example, tablets or capsules prepared by conventional means with pharmaceutically acceptable excipients, such as binding agents (for example, pregelatinized maize starch, polyvinylpyrrolidone, or hydroxypropyl methylcellulose); fillers (for example, lactose, microcrystalline cellulose, or calcium hydrogen phosphate); lubricants (for example, magnesium stearate, talc, or silica); disintegrants (for example, potato starch or sodium starch glycolate); or wetting agents (for example, sodium lauryl sulfate). The tablets can be coated by methods well known in the art. Solid dosage forms for oral administration include, but are not limited to, capsules, tablets, pills, powders, and granules. In such solid dosage forms, the active compounds are mixed with at least one pharmaceutically acceptable excipient or carrier such as, but not limited to, sodium citrate or dicalcium phosphate. Liquid preparations for oral administration can take the form of, for example, solutions, syrups, or suspensions, or they can be presented as a dry product for constitution with water or other suitable vehicle before use. Such liquid preparations can be prepared by conventional means with pharmaceutically acceptable additives such as suspending agents (such as sorbitol syrup, cellulose derivatives, or hydrogenated edible fats); emulsifying agents (such as lecithin or acacia); non-aqueous vehicles (such as almond oil, oily esters, ethyl alcohol, or fractionated vegetable oils); and preservatives (such as methyl or propyl-p-hydroxybenzoates or sorbic acid). The preparations can also contain buffer salts, flavoring, coloring, and sweetening agents as appropriate. For solid compositions, conventional non-toxic solid carriers can include pharmaceutical grades of mannitol, lactose, starch, or magnesium stearate. Actual methods of preparing such dosage forms are known, or will be apparent, to those of ordinary skill in the art. Oral administration includes buccal or “sub-lingual” administration via membranes of the mouth. This can be accomplished using lozenges or a chewable gum.

Pharmaceutical compositions suitable for oral administration can be presented in discrete units each containing a predetermined amount of at least one therapeutic compound useful in the present methods; as a powder or granules; as a solution or a suspension in an aqueous or non-aqueous liquid; or as an oil-in-water or water-in-oil emulsion. As indicated, such compositions can be prepared by any suitable method of pharmacy, which includes the step of bringing into association the active compound(s) and the carrier (which can constitute one or more accessory ingredients). In general, the compositions are prepared by uniformly and intimately admixing the active compound with a liquid or finely divided solid carrier, or both, and then, if necessary, shaping the product.

For example, a tablet can be prepared by compressing or molding a powder or granules of the compound, optionally with one or more accessory ingredients. Compressed tablets can be prepared by compressing, in a suitable machine, the compound in a free-flowing form, such as a powder or granules optionally mixed with a binder, lubricant, inert diluent, or surface active/dispersing agent(s). Molded tablets can be made by molding, in a suitable machine, the powdered compound moistened with an inert liquid diluent.

Solid compositions of a similar type can also be employed as fillers in soft and hard filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They can optionally contain opacifying agents and can also be of a composition that they release the active ingredient(s) in a certain part of the intestinal tract, optionally, in a delayed manner. Examples of embedding compositions which can be used include polymeric substances and waxes. The active compounds can also be in micro-encapsulated form, if appropriate, with one or more of the above mentioned excipients.

Liquid dosage forms for oral administration include, but are not limited to, pharmaceutically acceptable emulsions, solutions, suspensions, syrups, teas, and elixirs. In addition to the active compounds, the liquid dosage forms can contain inert diluents commonly used in the art such as, for example, water or other solvents, solubilizing agents, and emulsifiers, such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents. In some embodiments, suspensions, in addition to the active compounds, can contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

A drinkable tea can also be used in the present methods. A drinkable tea may be taken in a liquid form or in a once pulverized or granulated form together with water or hot water. When it is in a powdery or granular form, the drinkable tea may be contained in a cavity of mouth before taking hot water or water like the conventional powdery or granular drinkable tea, or it may be taken after once dissolving in hot water or water. One or more components, such as a sugar, mint, or other flavor, can be added to improve taste and easiness as a drinkable drug. Teas, syrups, and elixirs can be formulated with sweetening agents, for example glycerol, sorbitol, or sucrose. Such compositions can also contain a demulcent, a preservative, and flavoring and coloring agents.

Optionally, the pharmaceutical composition includes a parenteral carrier, and, in some embodiments, it is a solution that is isotonic with the blood of the recipient. Examples of such carrier vehicles include water, saline, Ringer's solution, and dextrose solution. Non-aqueous vehicles, such as fixed oils and ethyl oleate, are also useful herein, as well as liposomes.

The pharmaceutical compositions may be in the form of particles comprising a biodegradable polymer or a polysaccharide jellifying or bioadhesive polymer, an amphiphilic polymer, an agent modifying the interface properties of the particles and a pharmacologically active substance. These compositions exhibit certain biocompatibility features which allow a controlled release of the active substance. (See U.S. Pat. No. 5,700,486, incorporated herein by reference in its entirety).

In some embodiments, a PNPase inhibitor or a PNPase purine nucleoside substrate is included in a controlled release formulation, for example, a microencapsulated formulation. Various types of biodegradable and biocompatible polymers can be used, and methods of encapsulating a variety of synthetic compounds, proteins, and nucleic acids, have been well described in the art (see, for example, U.S. Patent Publication Nos. 2007/0148074; 2007/0092575; and 2006/0246139; U.S. Pat. Nos. 4,522,811; 5,753,234; and 7,081,489; PCT Publication No. WO/2006/052285; Benita, Microencapsulation: Methods and Industrial Applications, 2nd ed., CRC Press, 2006, all of which are incorporated by reference herein in their entireties).

In other embodiments, PNPase inhibitor or PNPase purine nucleoside substrate is included in a nanodispersion system. Nanodispersion systems and methods for producing such nanodispersions are well-known to one of skill in the art. (See, for example, U.S. Pat. No. 6,780,324; U.S. Pat. Publication No. 2009/0175953, both of which are incorporated herein by reference in their entireties). For example, a nanodispersion system includes a biologically active agent and a dispersing agent (such as a polymer, copolymer, or low molecular weight surfactant). Exemplary polymers or copolymers include polyvinylpyrrolidone (PVP), poly(D,L-lactic acid) (PLA), poly(D,L-lactic-co-glycolic acid (PLGA), poly(ethylene glycol). Exemplary low molecular weight surfactants include sodium dodecyl sulfate, hexadecyl pyridinium chloride, polysorbates, sorbitans, poly(oxyethylene) alkyl ethers, poly(oxyethylene) alkyl esters, and combinations thereof. In one example, the nanodispersion system includes PVP and PNPase inhibitor or PNPase purine nucleoside substrate (such as 80/20 w/w). In some examples, the nanodispersion is prepared using the solvent evaporation method (see, for example, Kanaze et al., Drug Dev. Indus. Pharm. 36:292-301, 2010; Kanaze et al., J. Appl. Polymer Sci. 102:460-471, 2006, both of which are incorporated herein by reference in their entireties).

Many types of release delivery systems are available and known to those of ordinary skill in the art. They include polymer based systems, such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109 (incorporated herein by reference in its entirety). Delivery systems also include non-polymer systems, such as lipids, including sterols such as cholesterol, cholesterol esters and fatty acids or neutral fats, such as mono-, di-, and tri-glycerides; hydrogel release systems; silastic systems; peptide-based systems; wax coatings; compressed tablets using conventional binders and excipients; partially fused implants; and the like. Specific examples include, but are not limited to, (a) erosional systems in which a PNPase inhibitor or a PNPase purine nucleoside substrate is contained in a form within a matrix, such as those described in U.S. Pat. Nos. 4,452,775; 4,667,014; 4,748,034; 5,239,660; and 6,218,371 (all of which are incorporated by reference herein in their entireties) and (b) diffusional systems in which an active component permeates at a controlled rate from a polymer, such as described in U.S. Pat. Nos. 3,832,253 and 3,854,480 (both of which are incorporated by reference in their entireties). In addition, pump-based hardware delivery systems can be used, some of which are adapted for implantation.

Use of a long-term sustained release implant may be particularly suitable for treatment of age-related retinal disease. Long-term release, as used herein, means that the implant is constructed and arranged to deliver therapeutic levels of the active ingredient for at least 30 days, and preferably 60 days. Long-term sustained release implants are well-known to those of ordinary skill in the art and include some of the release systems described above. These can be introduced, for example, via ocular delivery. In some embodiments, administration is from an ocular delivery device that is specifically adapted for delivery to the eye or a subcompartment thereof. For example, the device can be made of a sterile and/or biologically inert material configured for implantation in the eye. The device may use an internal reservoir (for example, from an implant disposed at an intra- or extra-ocular location (see, U.S. Pat. Nos. 5,443,505 and 5,766,242)) or from an external reservoir (for example, from an intravenous bag). A variety of devices suitable for administering components locally to the inside of the eye are known in the art. See, for example, U.S. Pat. Nos. 6,251,090, 6,299,895, 6,416,777, 6,413,540, and PCT Publication No. PCT/US00/28187. Methods for administration of agents to the eye are known in the medical arts and can be used to administer components described herein.

In some examples, the compositions disclosed herein can be administered with another treatment or composition. For example, in some embodiments, the compositions herein can be administered with low vision rehabilitation, implanting a telescopic lens, bevacizumab (AVASTIN®), ranibizumab (LUCENTIS®), aflibercept (EYLEA®), photodynamic therapy, and/or photocoagulation, such as for subjects that have AMD. In some embodiments, the compositions herein can be administered with prostaglandins (for example, latanoprost (XALATAN®), travoprost (TRAVATAN Z®), tafluprost (ZIOPTAN®), bimatoprost (LUMIGAN®), and latanoprostene bunod (VYZULTA®)), beta blockers (such as timolol (BETIMOL®, ISTALOL®, and TIMOPTIC®) and betaxolol (BETOPTIC®)), alpha-adrenergic agonists (such as apraclonidine (IOPIDINE®) and brimonidine (ALPHAGAN P®, QOLIANA®)), carbonic anhydrase inhibitors (such as dorzolamide (TRUSOPT®) and brinzolamide (AZOPT®)), rho kinase inhibitors (such as netarsudil (RHOPRESSA®)), miotic or cholinergic agents (such as pilocarpine (ISOPTO CARPINE®)), laser therapy (such as a laser trabeculoplasty), filtering surgery (such as a trabeculectomy), drainage tubes, and/or minimally invasive glaucoma surgery (MIGS), such as for subjects that have glaucoma.

### IV. Methods of Treating Age-Related Retinal Disease

Described herein are methods of treating age-related retinal disease in a subject. Methods are also disclosed for reducing inflammation and improving or restoring vasculature in a retina of a subject, such as subject with an age-related retinal disease. In some examples, the methods include selecting a subject with age-related retinal disease. In specific examples, the methods include selecting a subject with AMD, ganglion cell degeneration, glaucoma, LCA, RP, cone rod dystrophy, hypertensive retinopathy, RVO, CRAO, BRAO, or diabetic retinopathy. In some non-limiting examples, the subject can have AMD. The AMD can be wet or dry AMD. In other non-limiting examples, the subject has glaucoma. In some examples, selecting a subject includes diagnosis, such as with a full eye exam or a test for intra-ocular pressure. Any of the pharmaceutical compositions disclosed herein are of use in these methods. The pharmaceutical composition can be formulated for systemic or local delivery.

The subject can be a human or a veterinary subject. Veterinary subjects include domesticated animals or household pets, such as dogs, cats, horses, cows, and pigs. Non-human primates and wild animals can also be treated.

The subjects that can be treated using the methods herein can be of any age. However, generally, if untreated, an age-related retinal disease will worsen over time. The subject, such as a human subject, can be a child or an adult, for example, a younger adult, middle-aged adult, or older adult. For example, the subject can be at least about 1, at least about 2, at least about 5, at least about 10, at least about 12, at least about 16, at least about 18, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, or at least about 95, about 1-5, 5-10, 10-20, 20-30, 30-40, 40-50, 50-60, 60-70,70-80, 80-95, 1-20, 20-40, 40-60, 60-70, 70-80, 80-95, or at least about 50. In some examples, subjects include older adults, such as subjects at least about 40, 45, 50, 55, 60, 65, 70, or 85, or about 40-60, 45-55, 50-60, 50-70, or 50-65 years of age. In some examples, such as subjects with congenital defects (such as in subjects with LCA, cone rod dystrophy, or RP), the subjects can be children (such as 0-18 years old), young adults (such as about 18-25 years old), or older (such as 25-35, 25-45, or 30-40 years old or older). In one non-limiting example the subject is greater than 50 years of age. In other embodiment, the subject is greater than 60 years of age.

In some examples, the subject has a retinal disease. Retinal diseases include conditions in which the function or structure of the retina is damaged or decreased. In some examples, the subject has a retinal degenerative disease, such as a disease in which the retinal structure or function changes for the worse over time. In some examples, the subject has a retinal vascular diseases, such as eye diseases that affect the blood vessels in the eye. In some examples, the retinal disease is a degenerative and vascular disease. In specific examples, the subject can have LCA, RP, cone rod dystrophy, AMD, glaucoma, diabetic retinopathy, hypertensive retinopathy, RVO, CRAO, BRAO, diabetic retinopathy, or retinal detachment.

In specific, non-limiting examples, the subject can have AMD (such as caused by damage to the macula of the retina), including early, intermediate, or later AMD (such as wet or dry AMD). In particular examples, the methods herein can result in a decrease or delay in onset of AMD or symptoms thereof, such as decreased on delayed blurred or no vision in the center of the visual field in one or both eyes; difficulty with face recognition, reading, or other activities or in visual hallucinations. In particular examples, the subject with AMD is about 50 years or older. The subject can also have confounding genetic factors or be a smoker. Diagnosis can include a complete eye exam, and severity can range from early, intermediate, and late types, in which the late type can further include “dry” and “wet” forms.

In specific, non-limiting examples, the subject can have glaucoma (such as caused by damage the optic nerve. In particular examples, the methods herein can result in a decrease or delay in onset of glaucoma or symptoms thereof, such as patchy blind spots in the side (peripheral) or central vision and tunnel vision in advanced stages, such as in acute angle-closure glaucoma, or severe headache, eye pain, nausea and vomiting, blurred vision, halos around lights, or eye redness, such as in acute angle-closure glaucoma. In particular examples, the subject with glaucoma is about 60 years or older. Diagnosis can include a measuring intraocular pressure (tonometry), testing for optic nerve damage with dilated eye examination and imaging tests, examining for areas of vision loss (visual field test), measuring corneal thickness (pachymetry), and/or inspecting the drainage angle (gonioscopy).

In some examples, the methods include administering to the subject a therapeutically effective amount of a PNPase inhibitor or a PNPase purine nucleoside substrate, thereby treating the age-related retinal disease (such as a retinal disease, for example, AMD) in the subject. In some examples, the methods include administering to the subject a therapeutically effective amount of a PNPase inhibitor or a PNPase purine nucleoside substrate, thereby improving eye structure or function, such as retinal structure or function, for example, through improving or restoring vascular function or structure (for example, compared with a control subject without an age-related retinal disease) and decreasing inflammation. In some examples, administration of PNPase inhibitor or a PNPase purine nucleoside substrate can decrease symptoms or progression of the age-related retinal disease (such as a retinal disease, for example, AMD).

A variety of PNPase inhibitors or PNPase purine nucleoside substrates, such as those described herein, can be used in the methods. In some examples, a PNPase inhibitor is administered. In specific examples, a PNPase inhibitor includes guanine; guanosine; inosine; hypoxanthine; or guanine, guanosine, inosine, or hypoxanthine with a substituent at the 8-position, such as 8-substituted guanine, 8-substituted guanosine, 8-substituted inosine, or 8-substituted guanosine; a PNPase transition state analog (such as forodesine or a forodesine derivative, for example, DADMe-immucillin-H, DATMe-immucillin-H, or SerMe-immucillin-H); or a pharmaceutically acceptable salt thereof (such as a chloride salt, for example, a PNPase transition state analog chloride salt). Exemplary substituents (such as for 8-substituted guanine, 8-substituted guanosine, 8-substituted inosine, or 8-substituted guanosine) include amine, hydroxyl, nitro, nitroso, alkoxy, carbonyl, halogen, carboxyl, ester, carbonate, amide, or haloaliphatic. In specific examples, the substituent is amine. In specific, non-limiting examples, 8-substituted guanine, such as 8-AG can be used. In specific non-limiting examples, a PNPase transition state analog or a pharmaceutically acceptable salt thereof (such as a chloride salt) can be used. However, these are exemplary only. Any of the agents disclosed above are of use in these methods. The agent can be 8-AG.

A variety of administration modes can be used, as disclosed herein, such as oral administration, intravenous, or direct administration to the eye. In some examples, the PNPase inhibitor or a PNPase purine nucleoside substrate is administered to the subject once or more than once (such as repeatedly). In some examples, the PNPase inhibitor or PNPase purine nucleoside substrate is administered repeatedly, or one or more times (such as at least once, at least twice, at least three times, at least four times, at least five times, at least ten times, at least fifteen times, at least twenty times, at least thirty times, or more), such as one or more times daily, weekly, bimonthly, monthly, quarterly or per year. In other embodiments, the PNPase inhibitor or a PNPase purine nucleoside substrate is administered twice a day, daily, every other day, or 1, 2, 3, 4, 5, 6, or 7 times per week.

In some embodiments, the age-related retinal disease includes inflammation. For example, inflammatory cytokine expression can be increased, and administration of a therapeutically effective amount of a PNPase inhibitor or a PNPase purine nucleoside substrate decreases expression of the inflammatory cytokine. For example, the methods herein can include measuring ocular inflammation, such as by measuring nucleic acid expression of inflammatory cytokines. For example, amplification of inflammatory cytokine nucleic acid molecules can be used. In specific, non-limiting examples, the threshold cycle (Ct) method can be used (see, e.g., Schmittgen and Livak, Nature Protocols, 3(6): 1101-1108, 2008, incorporated by reference herein in its entirety). Expression of a control gene, such as one or more housekeeping genes, can also be measured through nucleic acid expression (such as through amplification of R-actin nucleic acid molecules, for example, using the Ct method). In some examples, the at least one inflammatory cytokine includes interleukin 1 beta (IL-1beta) and/or monocyte chemoattractant protein-1 (MCP-1). For example, measuring inflammatory cytokine nucleic acid expression can include using one or both of the following sets of primers:

(forward; SEQ ID NO: 1)
5′-GGGATGATGACGACCTGCTA-3′
and
(reverse; SEQ ID NO: 2)
5′-TGTCGTTGCTTGTCTCTCCT-3′,
such as to measure IL-1beta;
(forward; SEQ ID NO: 3)
5′-TGCAGAGACACAGACAGAGG-3′
and
(reverse; SEQ ID NO: 4)
5′-GCCAGTGAATGAGTAGCAGC-3′,
such as to measure MCP-1.

In specific examples, a housekeeping gene can also be measured, such as β-actin, for example, using:

(forward; SEQ ID NO: 5)
5′-ACTCTTCCAGCCTTCCTTC-3′
and
(reverse; SEQ ID NO: 6)
5′-ATCTCCTTCTGCATCCTGTC-3′.

In specific, non-limiting examples, the methods include using amplification (such as by the Ct method) to measure expression of at least one inflammatory cytokine nucleic acid molecule using primers encoded by SEQ ID NOS: 1-2 and/or 3-4 and to measure expression of a control nucleic acid molecule (such as 0-actin) in the subject using SEQ ID NOS: 5-6. In some examples, administration of a therapeutically effective amount of a PNPase inhibitor or a PNPase purine nucleoside substrate decreases the expression of IL-1beta and MCP-1 (such as measured using nucleic acid molecules, such as by Ct). For example, the expression of IL-1beta and MCP-1 can be decreased at least by 1-, 2-, 3-, 4-, 5-, 6-, 7-, 8-, 9-, 10-, 15-, or 20-fold, or about 1-2-fold, 1-5-fold, 1-10-fold, 1-20-fold, 5-10-fold, 5-15-fold, or 5-20-fold, or about 6- or 8-fold.

For retinal degeneration, diagnosis can utilize tests that examine the fundus of the eye and/or evaluate the visual field. These include electroretinogram, fluorangiography, and visual examination. The fundus of the eye examination aims to evaluate the condition of the retina and to evaluate for the presence of the characteristic pigment spots on the retinal surface. Examination of the visual field facilitates evaluation the sensitivity of the various parts of the retina to light stimuli. An electroretinogram (ERG) can be used, which records the electrical activity of the retina in response to particular light stimuli and allows distinct valuations of the functionality of the two different types of photoreceptors (i.e., cone cells and rod cells).

Following administration, the subject can be evaluated for response using any methods known in the art. These include, but are not limited to, ophthalomosccopy, perimetry, gonioscopy, pachymetry, or nerve fiber analysis. In some embodiments, retinal ganglion cell number and/or viability can be assessed. One of skill in the art can readily determine that the disclosed methods are effective. In particular embodiments, efficacy can be determined by whether the cup-to-disc ratio has stabilized. Scanning laser polarimetry or optical coherence tomography could be used, for example, to perform retinal nerve fiber layer analysis. A visual field test could be used to monitor progression of glaucoma. For any of the disclosed methods, therapeutic efficacy in treating a vision deficiency can manifest as an alteration in the individual's vision.

Measures of therapeutic efficacy will be applicable to the particular disease being modified and will recognize the appropriate detection methods to use to measure therapeutic efficacy. For example, therapeutic efficacy can be observed by fundus photography or evaluation of the ERG response. The method can include comparing test results after administration of the subject composition to test results before administration of the subject composition. As another example, therapeutic efficacy in treating a progressive cone dysfunction may be observed as a reduction in the rate of progression of cone dysfunction, as a cessation in the progression of cone dysfunction, or as an improvement in cone function, effects of which may be observed by, such as ERG and/or cERG; color vision tests; functional adaptive optics; and/or visual acuity tests, for example, by comparing test results after administration of the subject composition to test results before administration of the subject composition and detecting a change in cone viability and/or function. As another example, therapeutic efficacy in treating a vision deficiency can as an alteration in the individual's vision, such as in the perception of red wavelengths, in the perception of green wavelengths, in the perception of blue wavelengths, effects which may be observed by, cERG, and color vision tests, for example, by comparing test results after administration of the subject composition to test results before administration of the subject composition and detecting a change in cone and rod viability and/or function. In some embodiments, the method includes evaluation morphology and structure preservation and/or ERG.

The following examples are provided to illustrate particular features of certain embodiments, but the scope of the claims should not be limited to those features exemplified.

### EXAMPLES

Photoreceptor degeneration is a frequent event leading to blindness, occurring in conditions including retinitis pigmentosa (RP), other retinal dystrophies, age-related macular degeneration (AMD), and retinal toxicity. Other than the anti-vascular endothelial cell growth factor (anti-VEGF) treatments for wet AMD and the recent approval of Luxturna, a gene therapy product in a very rare form of congenital retinal dystrophy, Leber Congenital Amaurosis 2, there is currently no effective treatment available that can mitigate the rod or cone photoreceptor loss in RP or dry AMD. The high energy demand as reflected by the fact that the retina has the highest oxygen metabolism in the body, the constant stress from photochemical reactions, and the high content of unsaturated lipids make the retina a vulnerable tissue to oxidative stress. Thus, there is a need for efficacious agents treating photoreceptor degeneration caused by aging and genetic mutations. 8-AG is a potent and efficacious retinal protective agent in aged Fischer 344 (F344) rats and in the RHO^{P23H/+} knock-in mouse model of retinal degeneration. Systemic treatment with 8-AG showed reversal of the substantial age-related or genetic photoreceptor loss in these models. Only 8 weeks of daily oral treatments of 8-AG resulted in a distinctively thicker outer nuclear layer and outer segment (OS) layer in aged F344 rats. The strongly implied retinal protection also applied to RHO^{P23H/+} knock-in mice, where daily intraperitoneal injections of 8-AG significantly improved retinal function and also promoted a thicker outer nuclear layer after five weeks of treatment. These data indicate that the systemic administration of a purine analog can protect and reverse photoreceptor degeneration. 8-AG reduces retinal oxidative stress and inflammation and thereby prevents photoreceptor degeneration by promoting photoreceptor regeneration.

The findings disclosed herein reveal that 8-AG is a potent and efficacious retinal protective agent in two rodent models of retinal degeneration: aged Fischer 344 rats and RHO^{P23H/+} knock-in mice. The F344 rat has long been studied as a model of aging. As reported previously (Lai, et al., Invest. Ophthalmol. Vis. Sci. 17, 634-638 (1978); Lee et al., Vet. Pathol. 27, 439-444, doi:10.1177/030098589902700609 (1990); O'Steen & Landfield, Neurobiol. Aging 12, 455-462, doi:10.1016/0197-4580(91)90073-s (1991); DiLoreto et al., Brain Res. 647, 181-191, doi:10.1016/0006-8993(94)91316-1 (1994)), F344 rat retinae displayed age-related thinning of the outer nuclear layer (ONL) and outer/inner segment (OS/IS) layer that were more severe at the peripheral than in the central retinae, and such age-related photoreceptor loss was not seen in age-matched albino rats. Further, aged F344 rat retinae express elevated protein markers indicative of oxidative stress, whereas the levels of antioxidant proteins are decreased (Lenox, et al., Neurosci. Lett. 609, 30-35, doi:10.1016/j.neulet.2015.10.019 (2015)). The elevated pro-inflammatory marker chemokine ligand 5 and decreased anti-inflammatory cytokines such as interleukin 10 (IL-10) and IL13 suggest upregulated retinal immune responses. Together, these studies in F344 rats provide a profile of the temporal-spatial pathological progression of photoreceptor degeneration in a rodent model and reveal molecular events associated with age-related photoreceptor loss such as unfolded protein response, oxidative stress, and retinal immune responses.

The Rho^{P23H/+} knock-in mouse model is a well-studied rodent model of RP (Lobanova et al., Nat Commun 9, 1738, doi:10.1038/s41467-018-04117-8 (2018); Chiang et al., Mol. Neurobiol. 52, 679-695, doi:10.1007/s12035-014-8881-8 (2015); Athanasiou et al., Hum. Mol. Genet. 26, 305-319, doi:10.1093/hmg/ddw387 (2017)). Progressive rod loss undergoes a two-phase degeneration: the ONL thickness reduces to 50% at 1 month and is reduced to only one layer of nuclei at 9 months. The morphological thinning of the Rho^{P23H/+} retina is accompanied by its functional decline as revealed by the progressive decline of scotopic electroretinogram (ERG) responses, confirming rod cell death. Due to the inherent structural instability of the mutant rhodopsin (Chen, et al., J. Biol. Chem. 289, 9288-9303, doi:10.1074/jbc.M114.551713 (2014)), the Rho^{P23H/+} mouse retina shows an upregulated unfolded protein response (Chiang et al., Mol. Neurobiol. 52, 679-695, doi:10.1007/s12035-014-8881-8 (2015)), and the incompetence of misfolded rhodopsin degradation may contribute to rod cell death (Lobanova et al., Nat Commun 9, 1738, doi:10.1038/s41467-018-04117-8 (2018); Liu et al., FASEB J., doi:10.1096/fj.202000282R (2020); Dexter et al., eNeuro 7, doi:10.1523/ENEURO.0428-19.2019 (2020)). Similar to other rodent models of RP, the RNAseq and transcriptome analyses shows changed gene expression suggesting gliosis and retinal immune responses as early as 1 month of age. Taken together, the findings support that 8-AG, and other PNPase inhibitors or a PNPase purine nucleoside substrates, can reduce retinal oxidative stress and inflammation and thereby prevents photoreceptor degeneration and possibly promotes photoreceptor regeneration.

### Example 1

### 8-AG for Treating Age-Related Macular Degeneration

This example illustrates the use of a PNPase inhibitor (8-AG) for treating age-related macular degeneration (AMD) in an animal model.

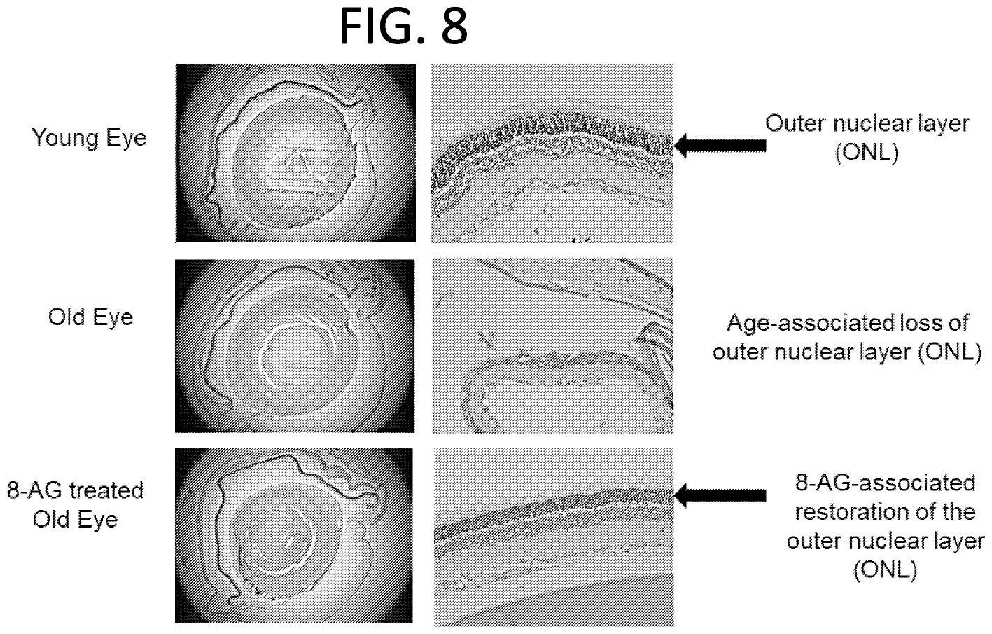

Aged Fischer 344 rats were treated with oral 8-AG (5 mg/kg/day for 6 weeks; drug delivered via the drinking water), or were a control, and the eyes of the rats were harvested for histological analysis of the retinal outer nuclear layer (ONL) and the results were compared with a corresponding histological analysis of young Fischer 344 rats (FIG. 8). With advance age, there is likely to be a loss of photoreceptors with associated thinning and eventual loss of the overlying ONL (see Schuman et al., Ophthalmology, 116:488-96.e2, 2009)

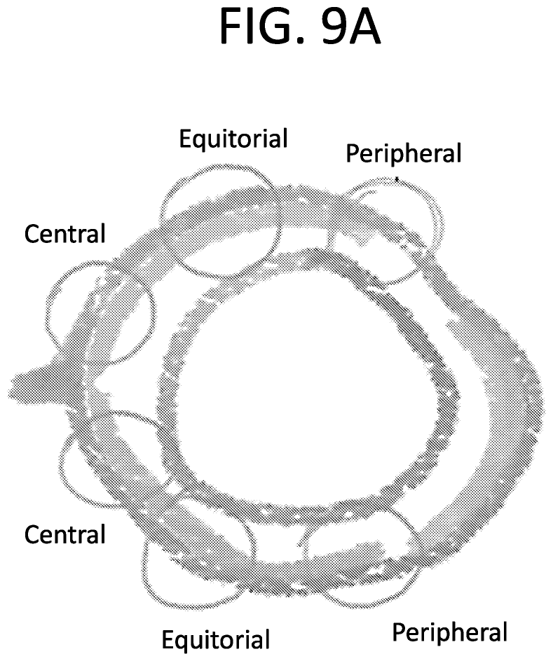

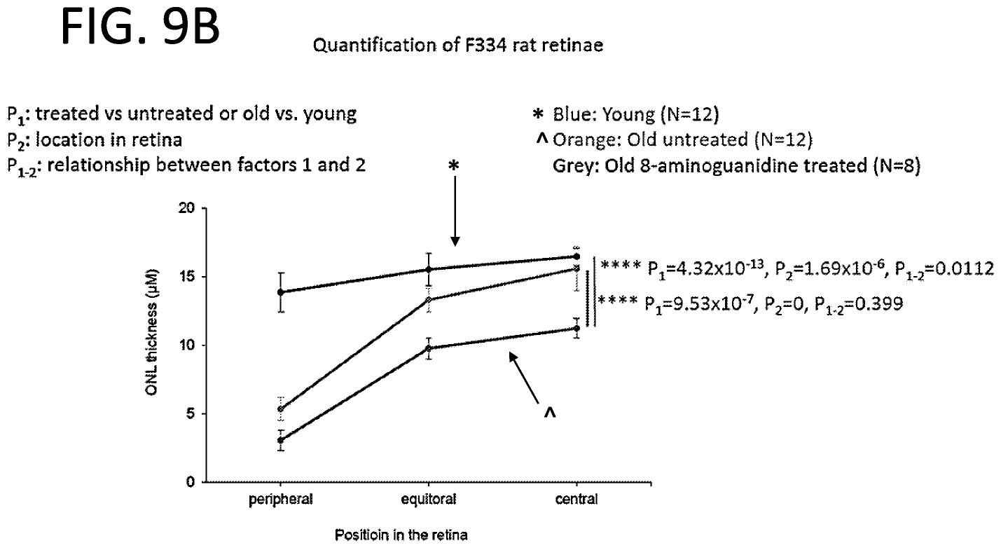

Photoreceptor degeneration was evident in the peripheral region of the old and untreated Fischer 344 rat retinae compared with the young retina controls. The ONL, which contains cell bodies of rods and cones, was further assessed on the edge of old retinae. The 8-aminoguanine-treated group showed ONL preservation in 7 out of 8 retinal edges, demonstrating a protective effect against age-related retinal degeneration. Quantification of the ONL thickness using a 2-way ANOVA confirms that the effect of treatment is significant, and this protection of photoreceptors does not depend on the location of the retina (FIG. 9). Taken together, these data indicate a benefit from 8-AG treatment in age-related retinal degeneration for not only the peripheral field, but also the entire visual field.

The following methods were used in the study presented in this example:

Histological analysis of the rat eyes: Rat eyes were marked at the superior side by a cautery pen and enucleated and fixed in 4% paraformaldehyde for over 24 h (1.5 years). The orientation of each eye was randomly embedded and sectioned. The fixed eyes were placed in a histology processing cassette for serial alcohol based dehydration. Dehydrated eyes were embedded in paraffin, and retinal cross sections were cut at 6 μm thickness. Retinal cross sections near the optic nerve were stained by hematoxylin and eosin (H&E). Images of H&E-stained retinal cross sections were collected under 4× and 20× objectives. The images at higher magnification (20×) were collected at the peripheral (tip of retina), equatorial (middle peripheral), and central (near optic nerve head) locations on both side of the optic nerve head for each rat retinal cross section (FIG. 9A).

In each retinal cross section image under the 20× objective, the outer nuclear layer (ONL) thickness was measured at 6 locations along the image to calculate the average ONL thickness. The ONL thickness represents the number of photoreceptors. The average ONL thickness at the central, equatorial, and peripheral locations from both sides of each cross section from each eye are listed in an Excel table, which was used to calculate the mean, SD, and SEM of ONL at the three locations.

Statistical analyses were performed using a two-way ANOVA, which compare treated vs untreated old rats or untreated old vs. young rats as the factor 1 and location of retinae as the factor 2 (FIG. 9B). P1<0.05 indicates a significant difference between treatment conditions or young vs. old animals. P2<0.05 indicates the ONL thickness varies at different locations of the retinae. P1-2<0.05 suggests that the treatment or age of the animals affected the ONL thickness with a dependency on the location of the retina.

### Example 2

### Potent Retinal Protection by Systemic Administration of 8-AG in the Animal Models of Age-Related Photoreceptor Degeneration and Retinitis Pigmentosa

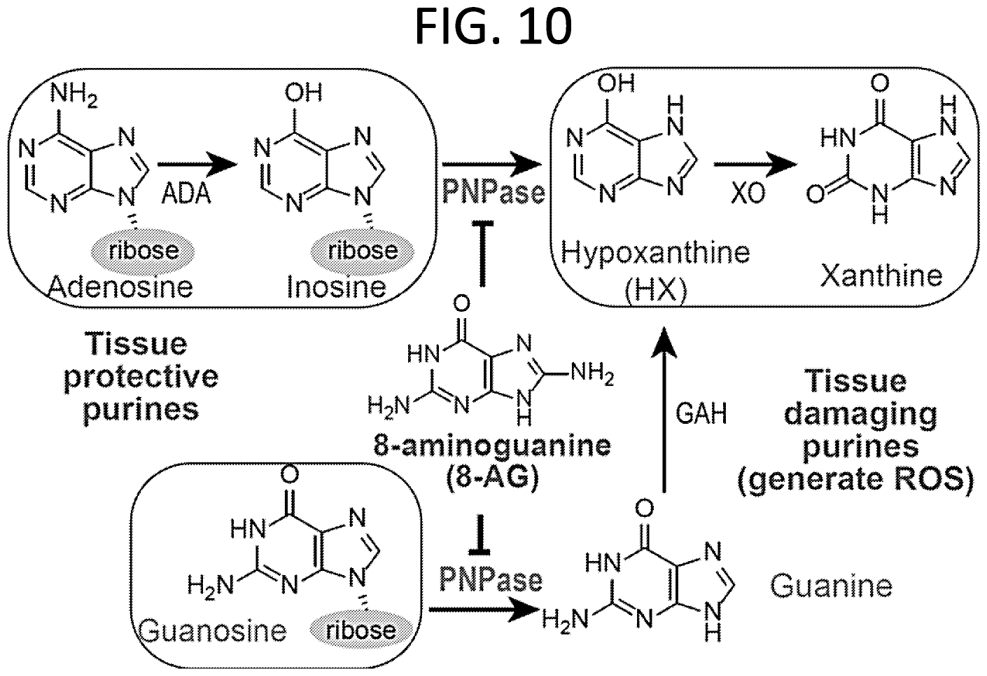

Changes in purine metabolism may relate to the extent of oxidative injury and cellular damage. Purine nucleoside phosphorylase (PNPase) belongs to the family of glycosyltransferases and is one of the key enzymes involved in the purine catabolic pathway (FIG. 10). PNPase transforms inosine and guanosine to their respective bases (i.e., inosine into hypoxanthine and guanosine into guanine). The PNPase substrates inosine and guanosine have been shown to provide beneficial anti-inflammatory and protective effects to various target organ systems including the lower urinary tract (LUT) (Liu et al., Urology 73, 1417-1422 (2009); Chung et al. PLoS One 10.137/0141492 (2015)) and the neural retina. Guanosine and adenosine are agonists of the A_{2A} receptor, which has been shown to positively improve RPE phagocytosis in mice (Liu et al., Ophthalmol Vis Sci 49, 772-780, doi:10.1167/iovs.07-0675 (2008); Guha et al., Exp. Eye Res. 126, 68-76, doi:10.1016/j.exer.2014.05.013 (2014)) and promote retinal regeneration in zebra fish (Guha et al., Exp. Eye Res. 126, 68-76, doi:10.1016/j.exer.2014.05.013 (2014)). Inosine has been reported to promote goldfish retinal ganglion cell axon outgrowth (Benowitz et al., Restor. Neurol. Neurosci. 19, 41-49 (2001)). In contrast, hypoxanthine, a product of PNPase, is tissue-damaging due to the production of ROS when metabolized by xanthine oxidase to xanthine (Willerns et al., Exp Gerontol 38, 1169-1177 (2003)). Pharmacologic inhibition of xanthine oxidase was shown to effectively improve the recovery of retinal function in the rat model of retinal ischemia (Roth, et al., Curr. Eye Res. 16, 875-885, doi:10.1076/ceyr.16.9.875.5045 (1997)). As a potent inhibitor of PNPase, 8-aminoguanine (8-AG) can increase the levels of ‘protective’ precursors (e.g., inosine and guanosine) while simultaneously decreasing levels of ‘toxic’ products (e.g., hypoxanthine and xanthine).

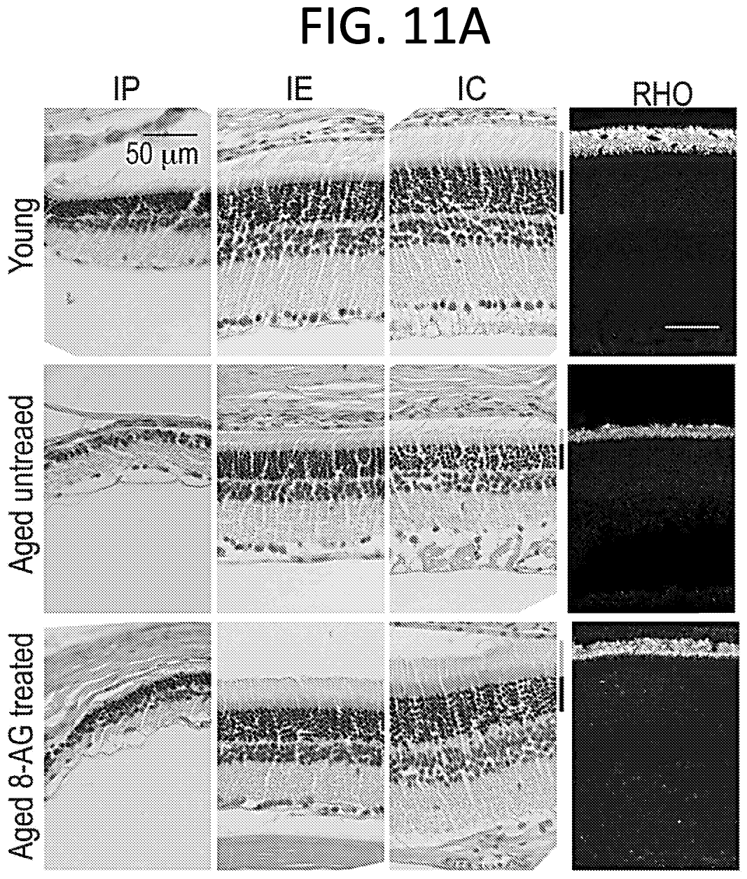

To test the efficacy of 8-AG in photoreceptor degeneration, 8-AG was administered in two animal models including the F344 rat of age-related photoreceptor degeneration, and the Rho^{P23H/+} knock-in mice of retinitis pigmentosa and examine the retinal morphology and function. F344 rats at 22-24 months of age showed substantial photoreceptor degeneration with significant thinner outer nuclear layer (ONL) and outer/inner segments (OS/IS) layer thicknesses throughout the retina, compared to young adult rats (FIGS. 11A-11D). Photoreceptor degeneration was more severe at the peripheral edge of the aged F344 retinae, and some animals showed complete loss of the ONL in that region (FIG. 11A). By treating the F344 rats with 5 mg per kg body weight (bw) per day of 8-AG in the drinking water for 8 weeks, starting from 22 months of age, distinctively increased ONL and OS/IS thickness was observed at the central, equatorial and peripheral retinal sites, compared to the untreated age-matched group (FIGS. 11A-11C). Rhodopsin levels were reduced in untreated aged retinae as reported (Lenox et 1., Neurosci. Lett. 609, 30-35, doi:10.1016/j.neulet.2015.10.019 (2015)), which were increased by 8-AG treatment (FIG. 11D), suggesting some functional restoration of treated retinae.

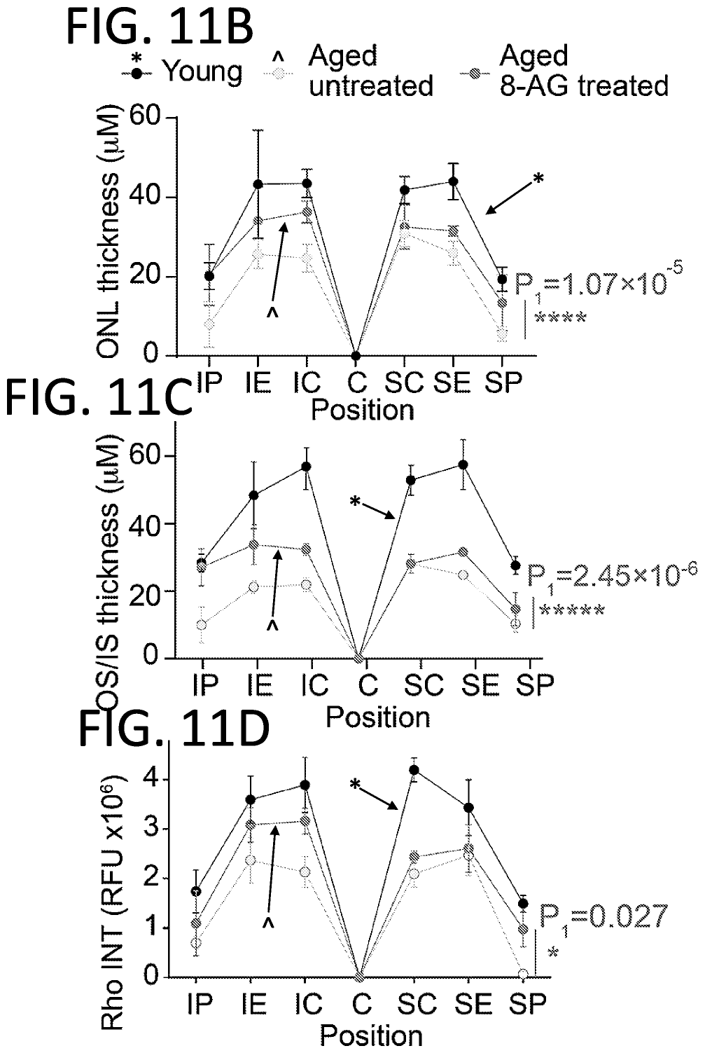

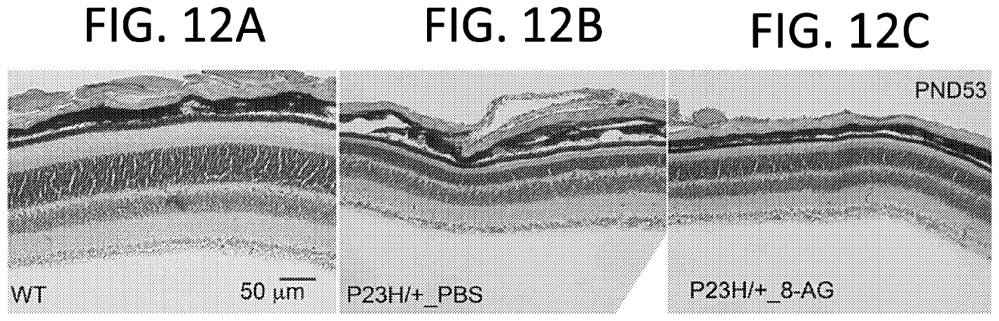

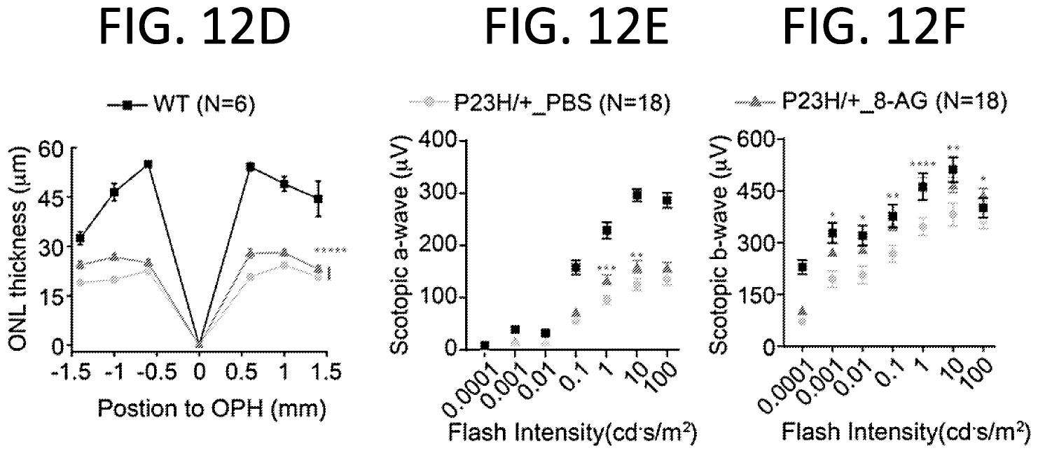

Similarly, strong retinal protection by 8-AG was observed in Rho^{P23H/+} knock-in mice (FIG. 12A-12F). By treating the mice with 11 mg per kg bw per day of 8-AG via intraperitoneal (i.p.) injections for five weeks starting from postnatal day (PND) 15, the scotopic electroretinogram (ERG) a- and b-wave responses were significantly higher than in the vehicle control group (FIGS. 12E, 12F), suggesting rod photoreceptor function was improved by 8-AG. The treated retinae also showed a thicker ONL layer (FIGS. 12A-12D), suggesting 8-AG protected photoreceptors from degeneration. Collectively, these results showed that systemic treatment with 8-AG protects and restores retinal morphology and function in age-related photoreceptor degeneration in F344 rats and also protects against rod photoreceptor death due to rhodopsin misfolding in the Rho^{P23H/+} knock-in mice.

### Example 3

### The Purine Degradation Pathway is Dysregulated in the Degenerative Retinae

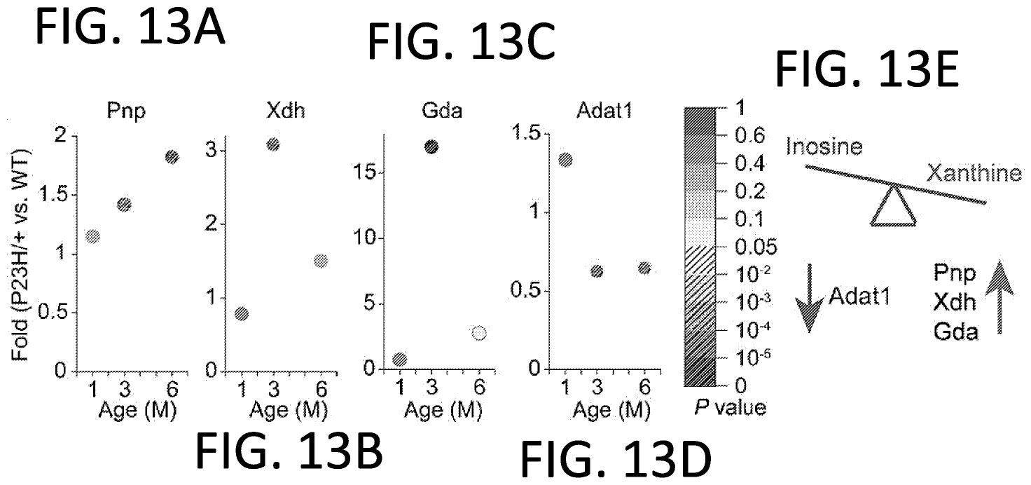

RNA-seq data comparing the transcriptome of Rho^{P23H/+} mouse retinae with WT control showed that the expression of enzymes in the purine degradation pathway was changed starting at 3 m of age. Significantly elevated expression of Pnp, Xdh, and Gda were observed in the Rho^{P23H/+} retinae compared to WT control (FIGS. 13A-13C). These genes express enzymes PNPase, xanthine dehydrogenase/oxidase and guanine deaminase that would cause the accumulation of tissue-damaging xanthine. In contrast, Adat1 was down-regulated in the Rho^{P23H/+} retinae after 3 months of age, and this gene expresses adenosine deaminase that produces the tissue-protective inosine (FIG. 13D). Consistent with the Rho^{P23H/+} model, expression of Xdh and Gda were also elevated in the aged F344 rat retinae, compared to young control. These findings suggest dysregulation of the purine degradation pathway favoring high levels of tissue-damaging xanthine and low levels of tissue-protective inosine (FIG. 13E) in the degenerative retinae.

### Example 4

### Metabolites of Guanosine, but not Adenosine, were Significantly Reduced in the Aged F344 Rat Retinae

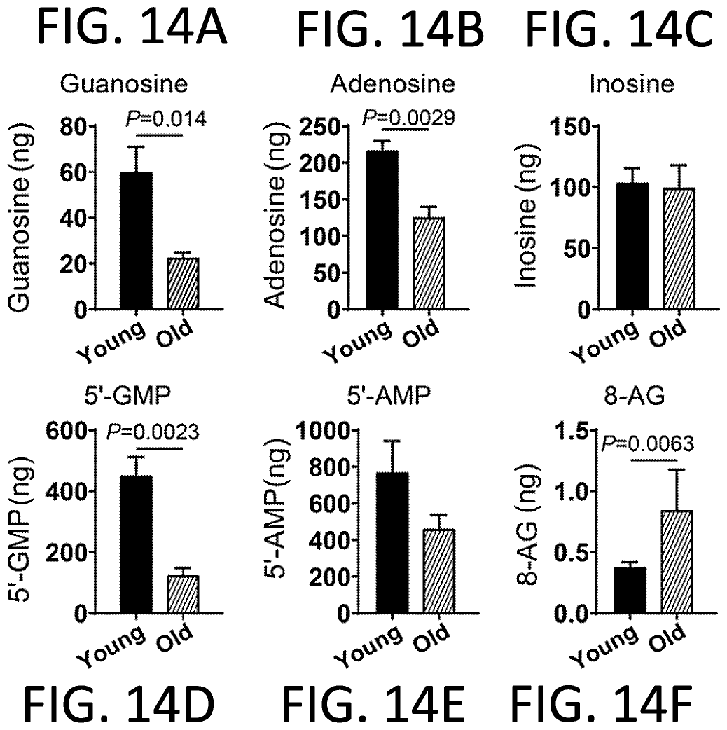

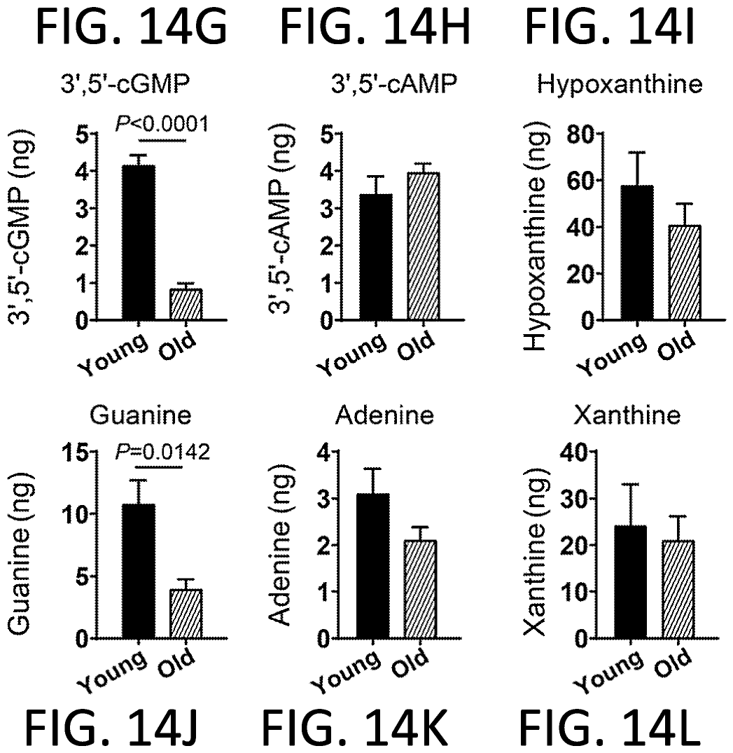

Using a triple quadrupole mass spectrometer operating in the selected reaction monitoring mode (ultra peroformance liquest chromatrograph—(UPLC)—mass spectrometry (MS)/MS), with methods developed previously, (Birder et al., JCI Insight 5, doi:10.1172/jci.insight.140109 (2020)), a shifted purine metabolome was detected in aged (22 m) vs. young (3 m) F344 rat retinae. While both the tissue-protective guanosine and adenosine were reduced in aged retinae (FIGS. 14A and 14B), the metabolites of guanosine including GMP, cGMP, and guanine were all significantly reduced (N=4, FIGS. 14A, 14D, and 14J), whereas the adenosine derivatives were not affected by age (FIGS. 14e, 14h, and 14K). This result showed that the metabolism of guanosine, but not adenosine, is correlated with age-related retinal degeneration in F344 rats. Because photoreceptor cells use cGMP as a second messenger for phototransduction, the observation that cGMP and its product GMP are substantially reduced in aged retinae is important. This dysregulation of the purine metabolome can contribute to the functional decline of photoreceptors that was observed by electroretinogram. The inosine level was not affected by aging (FIG. 14C). Surprisingly, it was found that 8-AG was increased ˜2 fold in aged F344 rat retinae (FIG. 14F). Without being bound by theory, this may explain why the PNPase products hypoxanthine and xanthines were not affected by aging (FIGS. 14F, 14I, and 14L). This result indicates a retinal-specific self-protective response that by elevating the biogenesis of 8-AG, tissue-damaging hypoxanthine and xanthine are maintained at a normal level.

### Example 5

### 8-AG Reversed Markers of Oxidative Stress and Mitochondrial Damage in the Aged F344 Rat Retinae

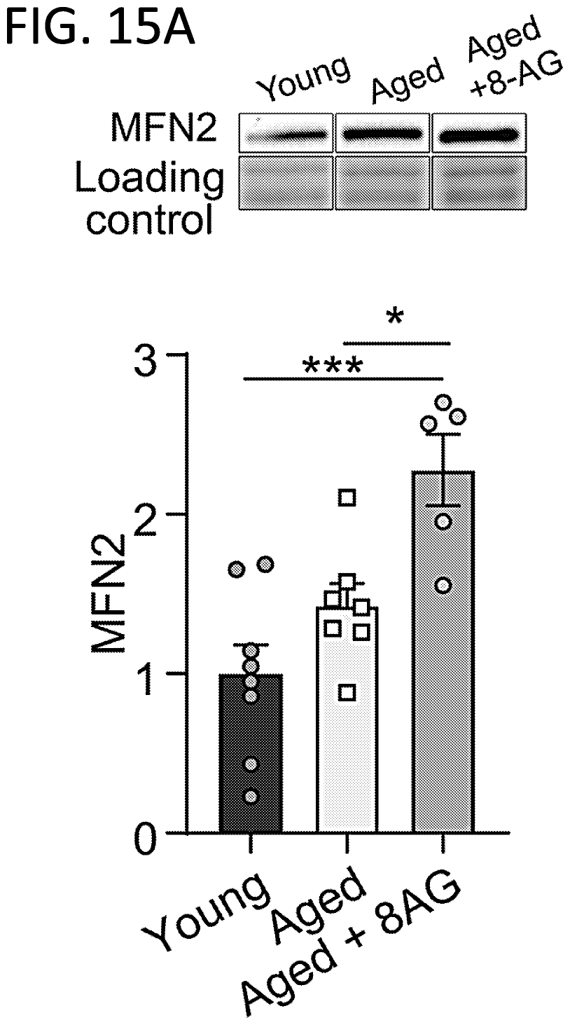

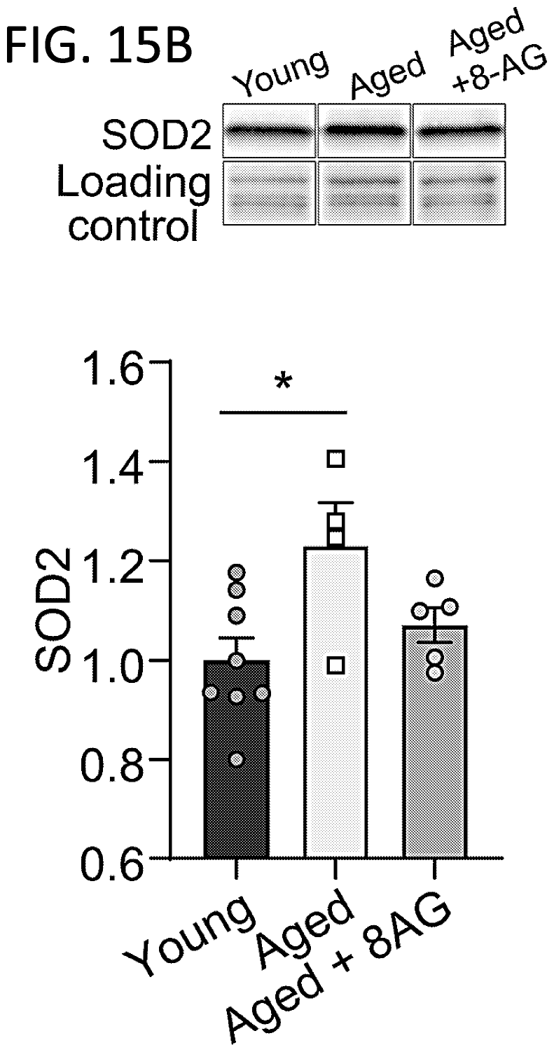

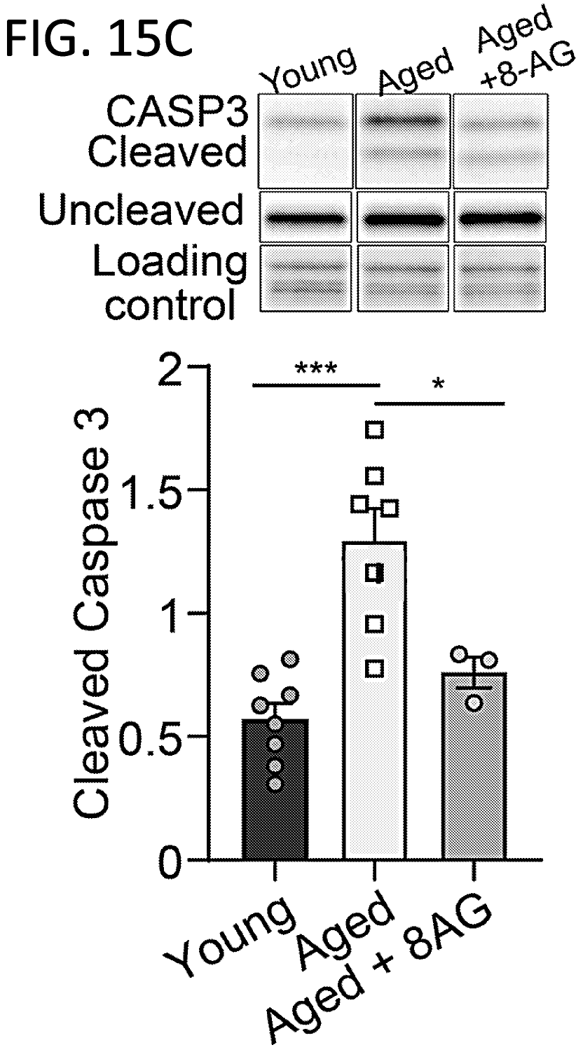

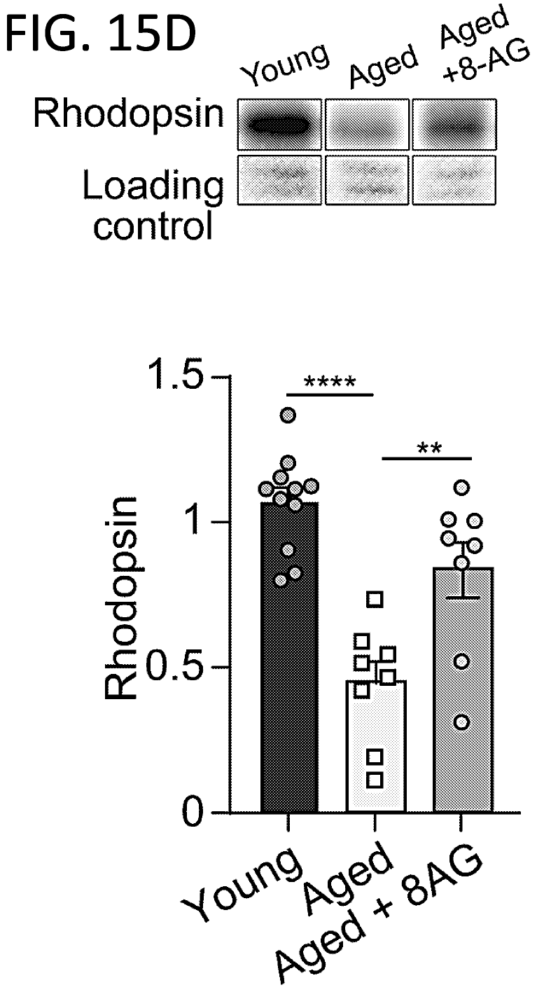

A range of protein markers of mitochondria function and apoptosis was probed in the retinae of young, aged and 8-AG treated aged F344 rat retinae, as well as the mitochondrial superoxide dismutase 2 (SOD2), an enzyme protecting tissue from damage by ROS, using immunoblots. It was found that retinal levels of mitofusin2 (MFN2), a protein involved in mitochondrial fusion, were not significantly affected by aging (FIG. 15A). These results suggest that the neural retina may be more resistant to age-related mitochondrial damage, which is consistent with our purine metabolome data showing higher levels of endogenous 8-AG in aged retinae. Nonetheless, administration of oral 8-AG (5 mg/kg) for 8 wks further increased retinal MFN2 levels, a finding which supports a protective effect of 8-AG against the development of mitochondrial dysfunction. In aged retinae, the levels of the antioxidant SOD2 were significantly increased (FIG. 15B); likely a response to counteract increased oxidative stress. The fact that chronic 8-AG decreased retinal levels of SOD2 is consistent with reduced retinal oxidative stress in 8-AG-treated aged rats. Caspase (CASP)3 cleavage was elevated twi fold in aged retinae (FIG. 15C); notably, 8-AG treatment reduced cleaved CASP3, which is evidence that 8-AG reduces apoptosis. Rhodopsin levels were substantially reduced in the aged F344 retina, which were restored by 8-AG treatment, confirming a functional restoration of the aged retinae by 8-AG (FIG. 15D). These results suggested that supplementing 8-AG increases mitochondrial homeostasis and reduces apoptosis.

### Example 6

### 8-AG Treatment Significantly Reduced the Number of Macrophages in the Aged F344 Rat Retinae

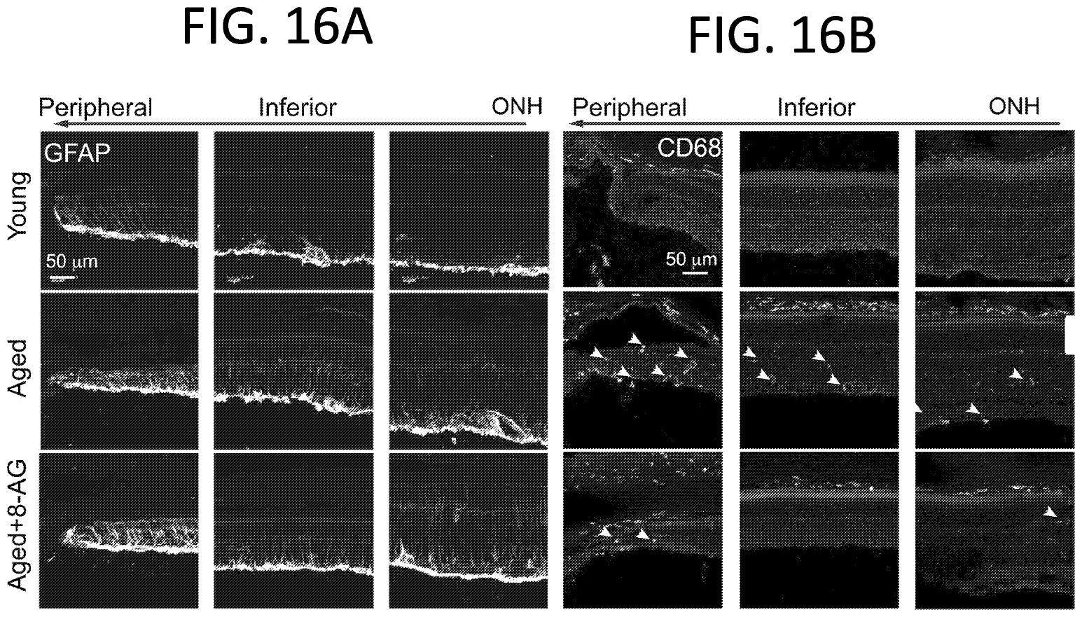

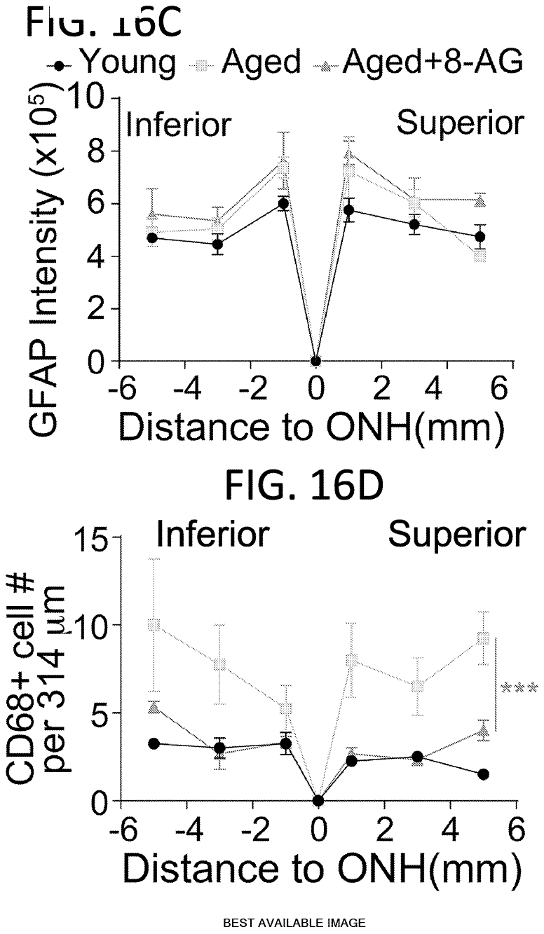

Glial cells are activated upon photoreceptor degeneration, and retinal immune response is thought to contribute to photoreceptor degeneration as the disease progresses. Activated macrophages were reported to not only scavenge dead cells, but also can phagocytose living photoreceptors (McMenamin et al. Prog. Retin. Eye Res. 70, 85-98, doi:10.1016/j.preteyeres. 2018.12.002 (2019)). Indeed, increased immunostaining of glial fibrillary acidic protein (GFAP) was observed in the aged F344 rat retinae, indicating activated Müller glia (FIGS. 16A and 16B). The Cluster of Differentiation 68+(CD68+) cells were also increased in the periphery of the aged retinae, suggesting an increased number of macrophages derived from microglia and/or infiltrated myeloid cells (FIGS. 16C and 16D). Interestingly, 8-AG treatment significantly reduced the number of CD68+ cells but did not affect GFAP staining. This result suggested that 8-AG treatment reduces the number of activated microglia/macrophages, but does not inhibit Müller glial activation.

### Example 7

### Transcriptome Analyses Indicate Oxidative Stress and Retinal Immune Responses in the Rho^{P23H/+} Mice

The RNA seq data comparing the 1-month-old Rho^{P23H/+} vs. age-matched WT mice showed 14 differentially expressed genes (DEGs) related to ROS metabolic processes (P=0.014), and 43 genes associated with positive regulation of immune responses (P=1×10⁻¹¹), indicating oxidative stress and retinal immune responses were activated as early as the onset of retinal degeneration. As degeneration progresses, more genes involved in these two pathways were differentially expressed in the Rho^{P23H/+} mouse retinae, compared to WT control at 3 months (48 DEGs in ROS metabolic process, P=3.5×10⁻⁹; and 125 DEGs in innate immune responses, P=2×10⁻³⁰) and 6 months of age (77 DEGs in ROS metabolic process, P=4.4×10⁻¹²; 182 DEGs in blood vessel morphogenesis, P=1.8×10⁻³⁰; and 162 DEGs in response to cytokines, P=6.5×10⁻²¹). These data confirm that oxidative stress and inflammation are exacerbated as retinal degeneration getting advanced in the Rho^{P23H/+} mice.

### Example 8

### ONL Thickness and Scotopic ERG Responses were Increasing in the Aged F344 Rat Retinae Treated with 8-AG

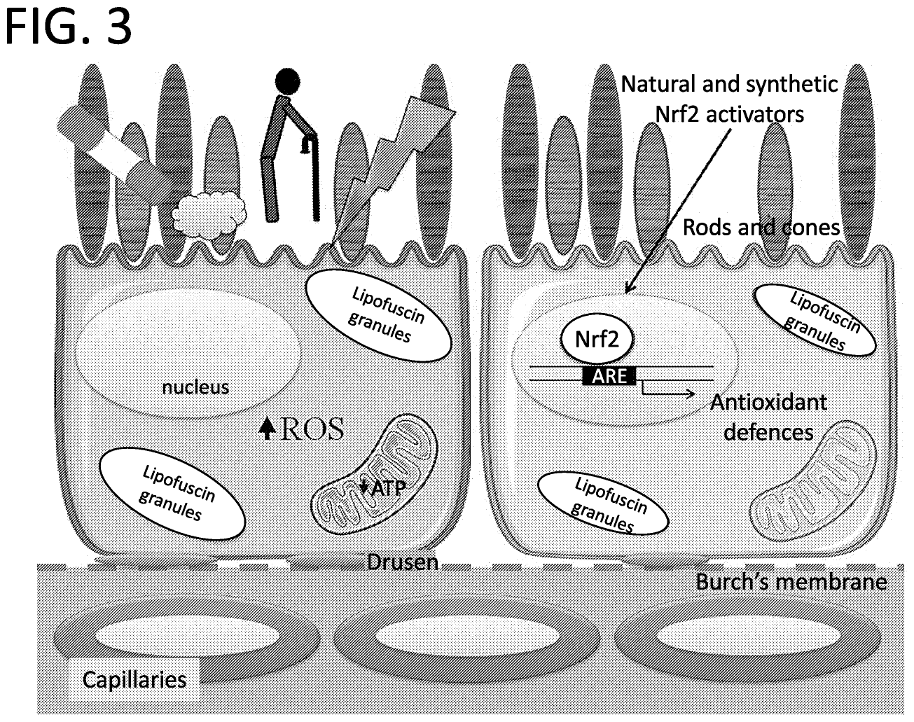

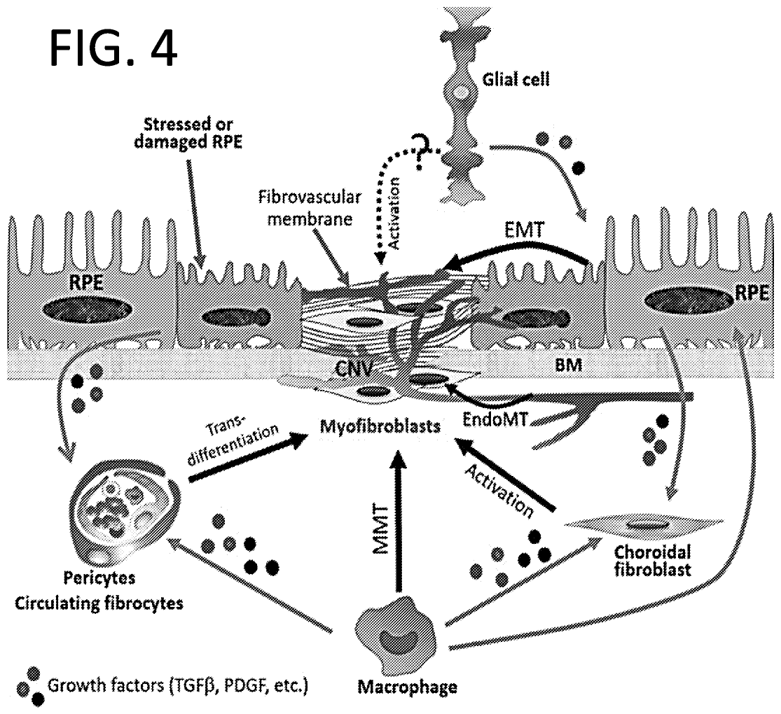

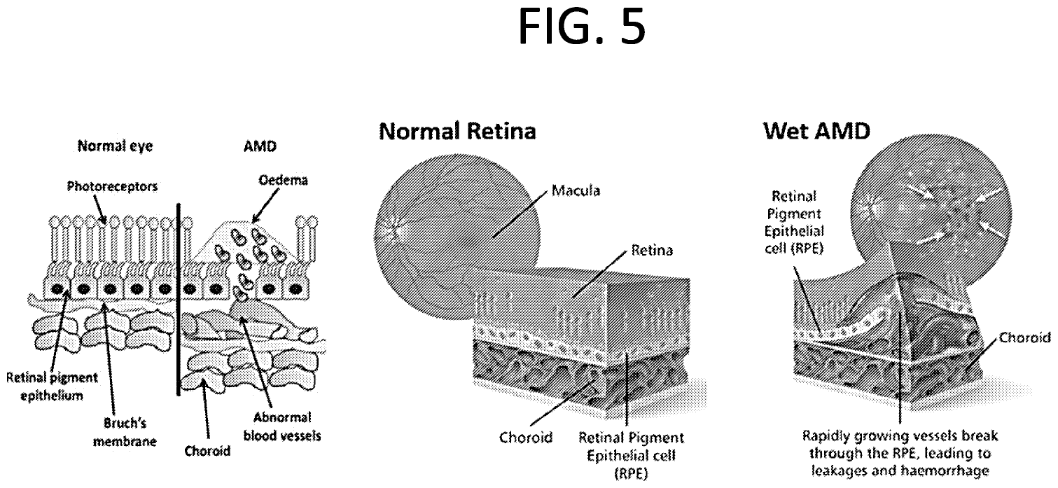

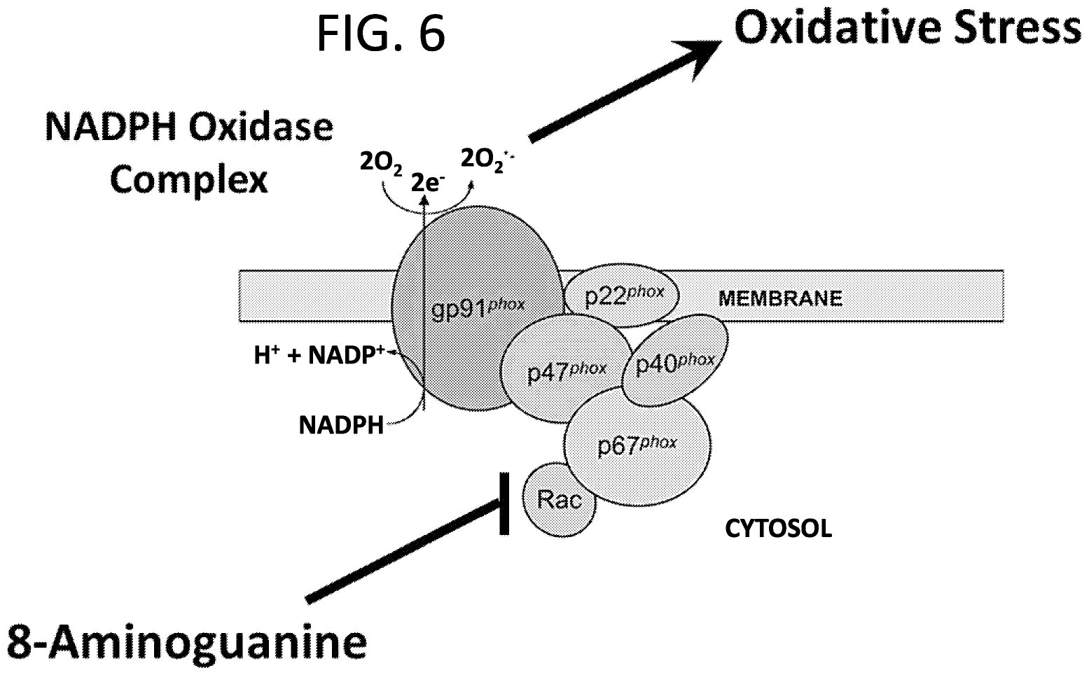

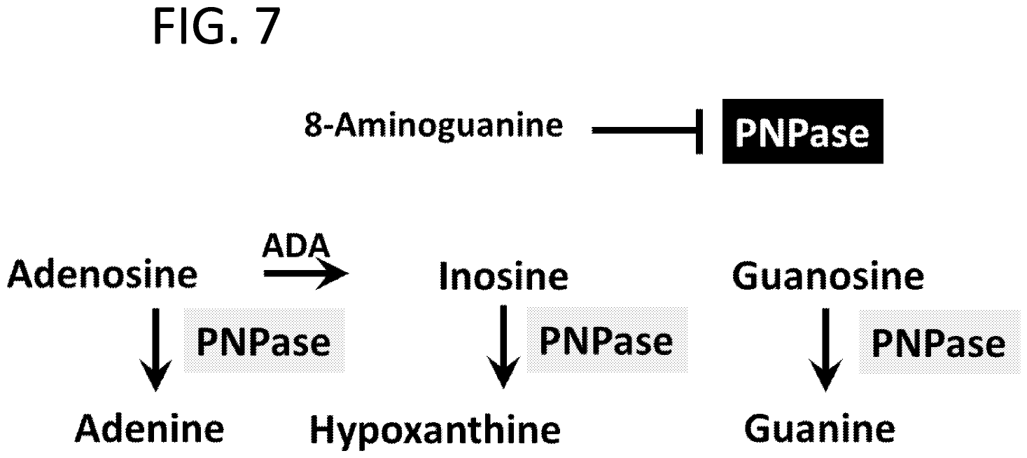

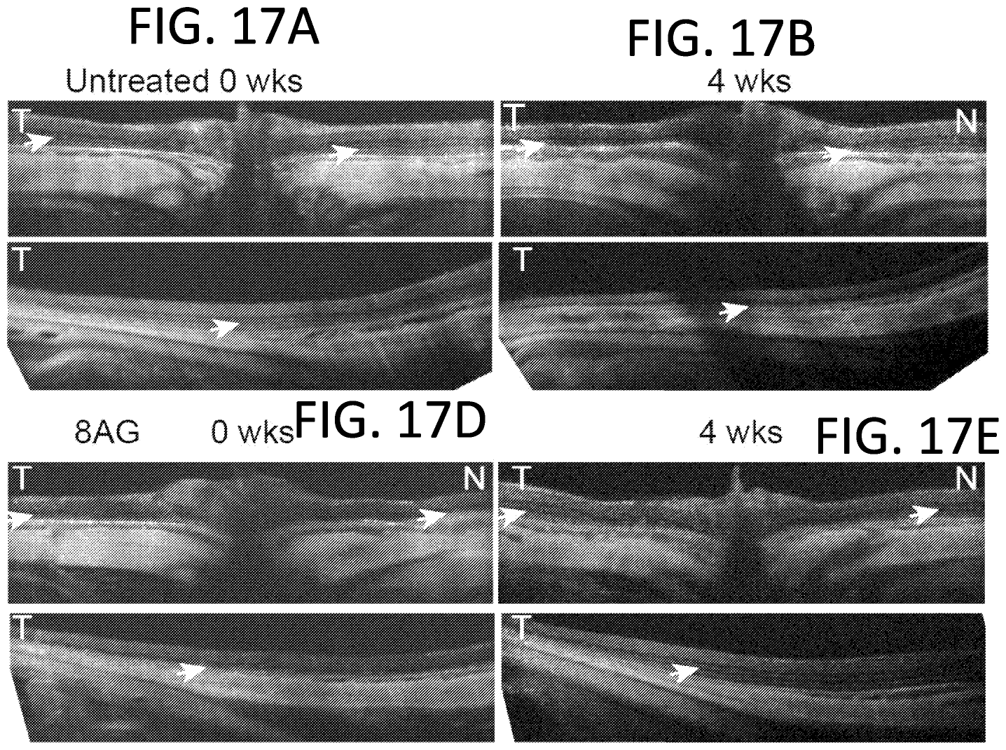

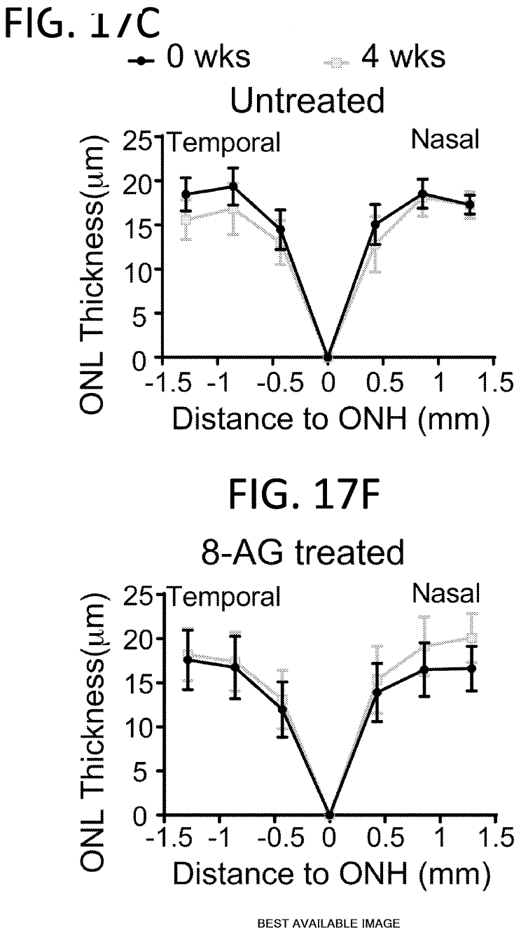

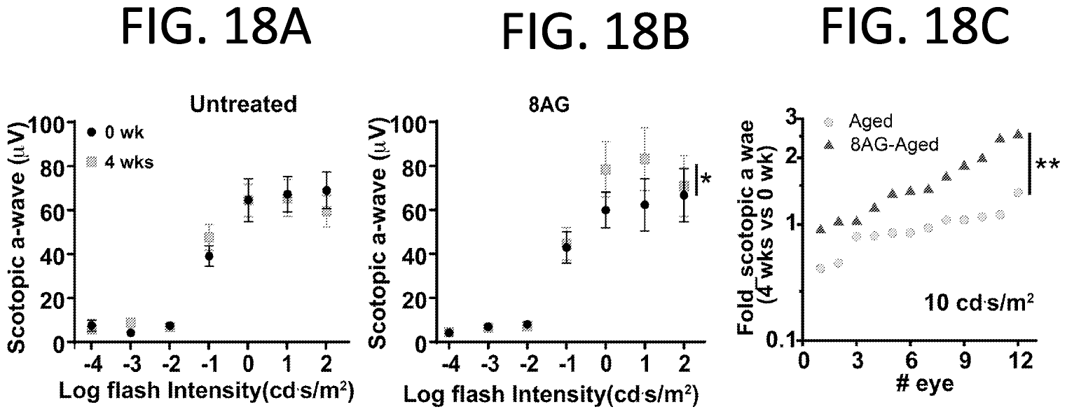

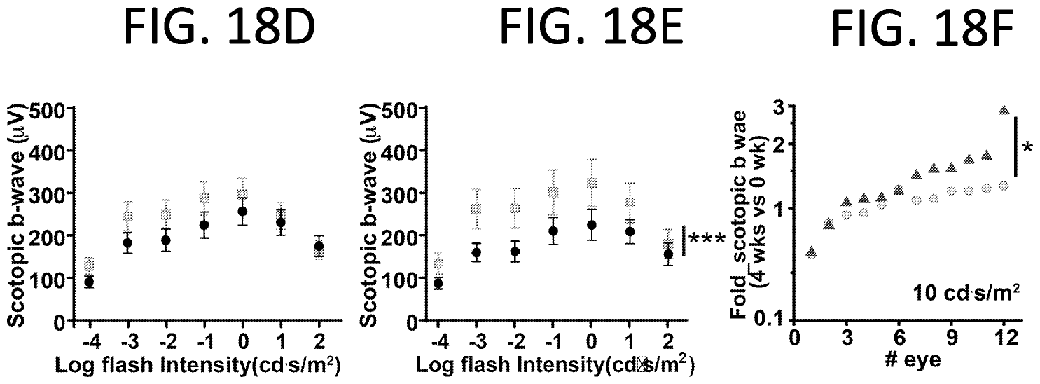

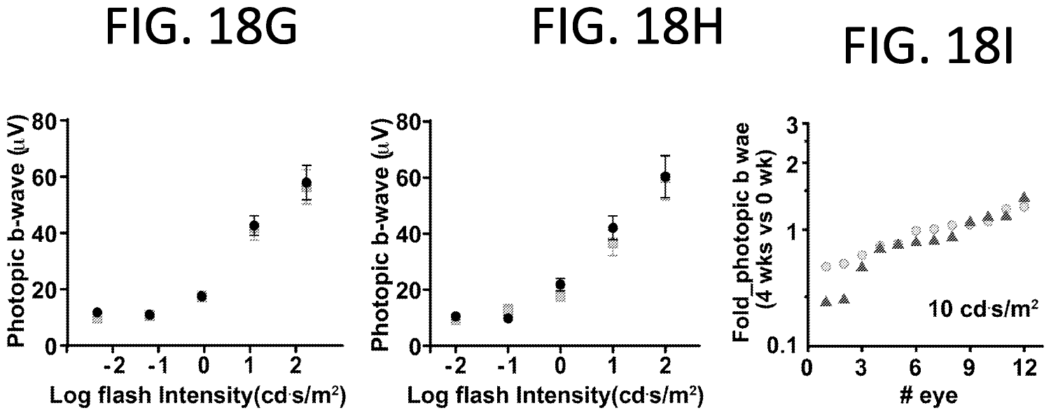

The potent efficacy of 8-AG in preserving retinal morphology and function of the aged F344 rats and Rho^{P23H/+} mice were shown in FIGS. 11A-11D and 12A-3F. By following the temporal changes of retinal morphology and function in the same group of aged F344 rats (22-24 m) via SD-OCT (FIGS. 17A-17E) and ERG (FIG. 18), it was demonstrated that 8-AG treatment not only delay photoreceptor loss, but even boosted retinal morphology and function. Within the 4 wks time (24 to 25 m), the ONL thickness of untreated aged rat retinae did not change except for a small decline on the temporal side (FIG. 17C), whereas the 8-AG treated retinae showed no change on the temporal side and slight increase on the nasal side at 4 wks, compared to before treatment (FIG. 17F). More strikingly, following the changes of a single exemplary retina, while the untreated retina showed time-dependent degeneration (FIGS. 17A and 17B), the 8-AG treated retina showed multiple sites where ONL disappeared before treatment and reappeared at 4 wks of treatment (FIGS. 17D and 17E). Accordingly, no changes were seen in scotopic and photopic ERG responses in the untreated aged rat from 0 to 4 wks, whereas 8-AG treatment significantly increased the scotopic a- and b-waves at 4 wks vs. 0 wk (FIGS. 18A-18F), but did not affect the photopic b wave responses (FIGS. 18G-18I), suggesting that 8-AG treatment improved rod function in the aged F344 rat retinae. Collectively, these results suggest that 8-AG treatment not only preserved the surviving photoreceptors but may also stimulate the recovery of surviving photoreceptor cells or even trigger regeneration.

### Example 9

### The Safety Profile of 8-AG

8-AG, as an endogenous metabolite, showed no observed toxicity in the animal experiments that were performed. While the genetic loss of PNPase activity is toxic and results in a compromised immune system, partial inhibition of PNPase by pharmacological administration is safe. The chronic effects of 8-AG have been examined in Dahl salt-sensitive rats, which is a model of genetic hypertension that is exacerbated by providing a high salt diet. Dahl rats were provided a high salt diet with and without treatment with 8AG for 40 days and their brain images were taken by MRL MRI inages showed that 8-AG treated animals are protected from strokes in the cerebral cortex and subcortical areas seen in the untreated high-salt control (Jackson, et al., J. Neurochem. 141, 676-693, doi:10.1111/jnc.14018 (2017)). Next, organs from these animals were examined histologically by an independent veterinary pathology lab. No treatment-related microscopic alterations in the heart, kidney, liver, aorta, or adrenal gland in rats that received 8-AG. This result supports the conclusion that partial inhibition of PNPase is safe.

In summary, there was potent retinal protection by systemic administration of 8-AG in the aged F344 rats and Rho^{P23H/+} knock-in mice. 8-AG, by inhibiting PNPase, ameliorates oxidative stress and retinal immune responses, which resulted in increased photoreceptor survival and restored photoreceptor morphology and function. There was activity of 8-AG in aminals, not only at early stage of retinal degeneration, such as the RhoP23H/+ mice, but also in the advanced stage of photreceptor degeneration, such as the aged F344 rats. These advanced photoreceptor losses are common in human patients.

The same treatment regime also significantly increased the thickness of ONL and IS/OS layer in the aged F344 rat retinae, suggesting that 8-AG strongly protects photoreceptors. This study addressed the pharmacodynamics, pharmacokinetics and mechanism of actions of 8-AG and showed that inhibition of PNPase activity by 8-AG protects the degenerative retina by restoring the disturbed purine imbalance, mitigating oxidative stress, restoring mitochondrial abnormalities, and silencing overactive retinal inflammation.

While neural protective peptides (Di Pierdomenico et al., Ophthalmol. Vis. Sci. 59, 4392-4403, doi:10.1167/iovs.18-24621 (2018); Sieving et al., Proc. Natl. Acad. Sci. U.S.A 103, 3896-3901, doi:10.1073/pnas.0600236103 (2006); Garcia-Caballero et al., Mol. Vis. 24, 733-745 (2018); Sahel & Leveillard, Adv. Exp. Med. Biol. 1074, 499-509, doi:10.1007/978-3-319-75402-4_62 (2018)) and small molecules (Liu et al., FASEB J., doi:10.1096/fj.202000282R (2020); Chen et al., Nat Commun 9, 1976, doi:10.1038/s41467-018-04261-1 (2018); Phillips et al. Tauroursodeoxycholic acid preservation of photoreceptor structure and function in the rd10 mouselnvest. Ophthalmol. Vis. Sci. 49, 2148-2155, doi:10.1167/iovs.07-1012 (2008); Getter et al., J. Biol. Chem. 294, 9461-9475, doi:10.1074/jbc.RA119.008697 (2019); Bian et al., Sci. Rep. 7, 6015, doi:10.1038/s41598-017-06471-x (2017)) have shown variable efficacy in delaying retinal degeneration, they are typically administered at an early stage before substantial retinal degeneration occurs. Previously, no oral treatment has shown potent retinal protection at the mid or late stage of photoreceptor degeneration in any animal model. These advanced photoreceptor losses are common in patients, which emphasizes the potential benefit of the presently disclosed approach. In summary, a photoreceptor protective agent that can be orally administered and could eventually be tested in photoreceptor degenerative diseases including RP and AMD.

### Example 10

### Materials and Methods for Examples 2-10

Animals: Young and aged F344 rats were obtained from National Institute of Aging. Rho^{P23H/P23H} knock-in mice and wild type C57BL/6J were purchased from The Jackson Laboratory. Rho^{P23H/P23H} mice were crossed with the wild type (WT) mice animals to generate the heterozygous Rho^{P23H/+} which was used as a model of retinitis pigmentosa. housed and bred following Genotyping for Rho was conducted as guided (Sakami et al., J. Biol. Chem. 286, 10551-10567, doi:10.1074/jbc.M110.209759 (2011)) using forward and reverse primers: (i) GGT AGC ACT GTT GGG CAT CT (SEQ ID NO: 7); and (ii) GAC CCC ACA GAG ACA AGC TC (SEQ ID NO: 8), respectively. The PCR products at 573 and 399 bp indicated the P23H knock-in mutant and WT allele of the RHO gene, respectively. Mice and rats were bred and housed under standard 12-h light/12-h dark conditions.

For treatment, female F344 rats were orally administered with 5 mg per kg body weight (bw) of 8-aminoguanine (8-AG) per day in the drinking water starting at 22-24 months of age for 8 weeks. Non-treated age-matched F344 rats and young adult F344 rats at 3-4 months of age were used as controls. RhoP^{23H/+} mice were administered with 8-AG for 11 mg per kg bw per day by intraperitoneal injections starting from postnatal day (PND) 15 for five weeks. PBS treated Rho^{P23H/+} and WT mice were used as controls.

Electroretinogram (ERG): ERG was performed using the Celeris system (Diagnosys, Lowell, Mass., USA). Rats were placed in the dark overnight. During the ERG test, protect the rats from the light from the beginning to the finish of the scotopic test, only use the dim light to do the preparation in the dark room. Pupils were dilated with 1% tropicamide (Akorn, Lake Forest, Ill., USA) and 2.5% phenylephrine (Sigma). Rats were anesthetized with an intraperitoneal injection of ketamine at 80 mg/kg bw and xylazine at 7 mg/kg bw. Eyes were lubricated by a 0.3% hypromellose eye gel (Alcon, Fort Worth, Tex., USA). A heating pad was used to maintain body temperature at 37° C. Scotopic ERG responses of dark-adapted eyes to ten flashes from 0.0001 cd·s/m² to 100 cd·s/m² were recorded and averaged from three sweeps per flash intensity with inter-sweep intervals of 10 to 30 s. After exposed to 10 cd/m² illumination for 10 min, the photopic ERG responses were recorded from the light-adapted eyes in response to flashes from 0.01 cd·s/m² to 100 cd·s/m² in addition to a 10 cd/m² background light. P-values were calculated by a two-way ANOVA to determine the statistical significance between the response amplitudes in the 8-AG treated group and untreated group. Factor 1, treatment condition; and factor 2, flash intensity.

Optical coherence tomography (OCT): To assess the 8-AG treatment on old F344 rats, ultrahigh-resolution SD-OCT (Bioptigen, Morrisville, N.C.) was performed for in vivo imaging of rats retinas. Pupils were dilated with 1% tropicamide (Akorn, Lake Forest, Ill., USA) and 2.5% phenylephrine (Sigma). Rats were anesthetized with an intraperitoneal injection of ketamine at 80 mg/kg bw and xylazine at 7 mg/kg bw. Eyes were lubricated by a 0.3% hypromellose eye gel (Alcon, Fort Worth, Tex., USA). A heating pad was used to maintain body temperature; the A scan/B scan ratio was set at 1200 lines. Five frames of OCT images scanned at 0° were acquired in the B-mode, averaged, and saved as pdf files. To measure changes to photoreceptors in the retinas assess the effect of 8-AG on retinal protection, the thickness of the ONL was measured along with the scanned SD-OCT image at 6 points from the temporal to nasal end of the retina. A graph of ONL thicknesses was plotted to obtain the means and standard error (SE) of the samples.

Tissue collection and Immunohistochemistry (IHC): Rats were euthanized and a burn mark labeled the superior side of each eye with a Cautery pen. Eyes were enucleated by a surgical scissor cutting around the eyeball. Each eye was opened with a cut at the cornea side of the limbus, cut horizontally at the edge of the cornea for a circle, corneas removed and lens isolated. Eyecups were incubated in freshly prepared 4% paraformaldehyde for 2 hr. Fixed eyecups were dehydrated sequentially in 5, 10, 20 and 40% sucrose solutions in phosphate buffered saline (PBS) for 30 min each at room temperature. Finally, eyecups were incubated in a mixture of 40% sucrose in PBS and O.C.T. compound (FisherScientific, Houston, Tex. USA) at 3:7 volume ratio overnight at 4° C. before they are embedded in the same mixed solution in an orientation-specific manner and frozen in liquid nitrogen-bathed isobutene. A microtome made twelve-micron retinal cross-sections at −18° C. and those containing the optic nerve head were applied onto a SUPERFROST™ glass slide (FisherScientific, Houston, Tex. USA). These slides were then used for IHC.

After rehydration and permeabilization in PBST for 15 min, retinal sections were incubated in 5% goat serum for 30 min, and then they were incubated with 5% goat serum containing the primary antibodies. Mouse 1D4 anti-rhodopsin antibody (50 μg/mL) to stain the rhodopsin of the retina; rabbit CD68 antibody (ab125212, 1 μg/mL) to see the microglia in the retina; rabbit GFAP antibody (z0334, 6.4 μg/mL) to test the Müller glia of the retina. Hoechst 33342 (1:5000 dilution) was applied for 5 min to stain the nuclei. Sections were mounted with the PROLONGGOLD™ mounting solution (ThermoFisher, Waltham, Mass. USA). Immunofluorescence images were then taken by a confocal microscope for high-magnification images.

Retinal histology and H&E staining: To examine the structure of the aging rats' retinas treated with 8-AG, the rats were euthanized, their eyes removed, and fixed in 4% paraformaldehyde and 1% glutaraldehyde before paraffin sectioning. Paraffin sections (7 μm thick) were stained with H&E and imaged by light microscopy (Leica, Wetzlar, Germany).

RNA-seq and transcriptome analyses: Retinae were isolated freshly right after the animals were euthanized and total ribonucleic acid (RNA) was isolated by RNAZOl® as per manufacturer's instructions. Briefly, each retinae was lysed in 1 ml of RNAZOl® and mixed with 0.4 ml of nuclease-free water by vigorous vertex. The mixture was sedated for 15 min at room temperature before centrifuging at 12,000×g. The RNA containing top layer was gently taken out and mixed with equal volume of isopropanol and incubated at room temperature for 20 minutes, followed by centrifugation at 12000×g for 10 minutes, to precipitate the RNA. The pellet was washed twice with 70% ethanol followed by centrifugation at 7,000×g for 3 minutes each time. Finally, the clean RNA pellet dried was re-dissolved in 20 μl of nuclease-free water. The concentration and quality of purified total RNA was examined by a bioanalyzer. All the RNA samples with the RNA integrity (RIN) number more than seven were selected to prepared library, and the library size was analyzed using bioanalyzer before run for Hi-seq for RNAseq.

The reads were first mapped to the latest UCSC transcript set using Bowtie2 version 2.1.0 (Langmead and Salzberg, Nat Methods 9, 357-359, doi:10.1038/nmeth.1923 (2012)) and the gene expression level was estimated using RSEM v1.2.15 (Li and Dewey, BMC Bioinformatics 12, 323, doi:10.1186/1471-2105-12-323 (2011)). Differentially expressed genes were identified using the edgeR program (Robinson et al., Bioinformatics 26, 139-140, doi:10.1093/bioinformatics/btp616 (2010)). Genes showing altered expression with FDR<0.01 and more than 1.5 fold changes were considered differentially expressed. Goseq (Young et al., Genome Biol. 11, R14, doi:10.1186/gb-2010-11-2-r14 (2010) was used to perform the GO enrichment analysis and Kobas (Xie et al., Nucleic Acids Res. 39, W316-322, doi:10.1093/nar/gkr483 (2011)) was used to perform the pathway analysis. Panther and GSEA was used for over-represented pathways. Further analysis was performed using Ingenuity Pathways Analysis (IPA) and the annotated pathway gene were further analyzed using STRING: functional protein association networks for functional association.

Western Immunoblotting: The freshly isolated retinae were homogenized using Lysing Matrix D in a FASTPREP-24™ instrument (MP Biomedicals) in HBSS buffer (5 mM KCl, 0.3 mM KH₂PO₄, 138 mM NaCl, 4 mM NaHCO₃, 0.3 mM Na₂HPO₄, 5.6 mM glucose, and 10 mM HEPES, pH 7.4) containing complete protease inhibitor cocktail (1 tablet/10 ml, Roche) and phosphatase inhibitor cocktail (MilliporeSigma, 1:100). After centrifugation (16200 g, 15 minutes at 4° C.), the membrane protein fraction was prepared by suspending the membrane pellets in lysis buffer containing 0.3M NaCl, 50 mM Tris-HCl (pH 7.6), and 0.5% Triton X-100, as well as the same concentration of protease inhibitors as above. The suspensions were incubated on ice and centrifuged (16200 g, 15 minutes at 4° C.). The protein concentrations of the combined supernatants were determined using the Pierce BCA protein assay (Thermo Fisher Scientific). Two μg of unheated total protein was loaded on a 4%-15% TGX Stain-Free SDS-PAGE gel (Bio-Rad). As a reliable loading control, total protein measurement per sample was determined using Bio-Rad Stain Free SDS-PAGE gel technology. UV-activated protein fluorescence was imaged on a ChemiDoc MP (Bio-Rad). The gel was electrotransferred to polyvinylidene fluoride membrane and immunoblotted with 1D4 anti-rhodopsin antibody (5 μg/mL). After washing in TBS-T, the membranes were incubated with secondary antibody, sheep anti-mouse HRP (Southern Biotech), developed using WESTERNBRIGHT® Quantum (Advansta), and imaged on a ChemiDoc MP (BioRad). The volume (intensity) of each protein species was determined and normalized to total protein using Image Lab software (Bio-Rad).

Purine Metabolome Measurement: Half of each rat retina was collected and homogenized in 3 ml of ice-cold acetonitrile/methanol/water (1:2:2 v/v/v) using a glass homogenizer. The homogenized samples were heated in 60° C. water bath for 10 min to denature the enzymes in the retinae. Samples were cooled on ice and then stored at −80° C. before subsequent processing. Purines were extracted in the supernatant after centrifugation at 3,000×g for 90 minutes at 4° C. The remaining cell debris was extracted again by the addition of acetonitrile/methanol/water at a 1:2:2 volume ratio before a vigorous vortex and centrifugation at 3,000×g for 90 minutes at 4° C. The organic solvents from the purine extracts were dried by a speed vacuum concentrator and then the concentrated samples were diluted in water. Only 100 μL of ultra-filtered extracts were spiked with heavy-isotope internal standards and injected into reversed-phase liquid chromatography (Agilent ZORBAX® Eclipse XDB-C-18 column, 3.5 μm beads; 2.1×100 mm) to resolve purines which were sequentially quantified by a triple quadrupole mass spectrometer operating in the selected reaction monitoring mode (UPLC-MS/MS) with a heated electrospray ionization source and as previously described (Ren et al., J. Pharmacol. Exp. Ther. 328, 855-865, doi:10.1124/jpet.108.146712 (2009); Lai et al., Invest. Ophthalmol. Vis. Sci. 17, 634-638 (1978)). The following transitions (selected reaction monitoring) were obtained: guanosine (2844152 m/z, RT=3.10 min); ¹³C10⁵N₅-guanosine (299→162 m/z, RT=3.10 min); hypoxanthine (137→119 m/z, RT=1.86 min); ¹³C₅-hypoxanthine (142→124 m/z, RT=1.86 min); aminoguanine (167→150 m/z, RT=1.50 min); ¹³C₂¹⁵N-aminoguanine (170→153 m/z, RT=1.50 min).

In view of the many possible embodiments to which the principles of the disclosure may be applied, it should be recognized that the illustrated embodiments are only examples and should not be taken as limiting the scope. Rather, the scope is defined by the following claims. We therefore claim as our invention all that comes within the scope and spirit of these claims.

1. A method of treating a subject with an age-related retinal disease, comprising:
selecting the subject with the age-related retinal disease; and
administering to the subject a therapeutically effective amount of a purine nucleoside phosphorylase (PNPase) inhibitor and/or a PNPase purine nucleoside substrate, thereby treating the age-related retinal disease in the subject.

2. A method of reducing inflammation and improving or restoring vasculature in a retina of a subject, comprising:
selecting the subject, wherein the subject is in need of reduced inflammation and/or improved vasculature in the retina; and
administering to the subject a therapeutically effective amount of a purine nucleoside phosphorylase (PNPase) inhibitor and/or a PNPase purine nucleoside substrate,
thereby reducing inflammation and improving the vasculature in the retina of the subject.

3. The method of claim 1, wherein the PNPase inhibitor is a guanine comprising a substituent at the 8-position, a guanosine comprising a substituent at the 8-position, an inosine comprising a substituent at the 8-position, a hypoxanthine comprising a substituent at the 8-position, a PNPase transition state analog, or a pharmaceutically acceptable salt thereof.

4. The method of claim 3, wherein the substituent is amine, hydroxyl, nitro, nitroso, alkoxy, carbonyl, halogen, carboxyl, ester, carbonate, amide, or haloaliphatic.

5. The method of claim 3, wherein the substituent is amine.

6. The method of claim 3, wherein the guanine comprising a substituent at the 8-position is 8-aminoguanine.

7. The method of claim 3, wherein the PNPase transition state analog is:
7-[(2S,3S,4R,5R)-3,4-dihydroxy-5-(hydroxymethyl)pyrrolidin-2-yl]-3H,4H,5H-pyrrolo[3,2-d]pyrimidin-4-one;
7-(((3R,4R)-3-hydroxy-4-(hydroxymethyl)pyrrolidin-1-yl)methyl)-3H-pyrrolo[3,2-d]pyrimidin-4(5H)-one;
7-(((2R,3S)-1,3,4-trihydroxybutan-2-ylamino)methyl)-3H-pyrrolo[3,2-d]pyrimidin-4(5H)-one;
7-((1,3-dihydroxypropan-2-ylamino)methyl)-3H-pyrrolo[3,2-d]pyrimidin-4(5H)-one; or
a pharmaceutically acceptable salt thereof.

8. The method of claim 3, wherein the pharmaceutically acceptable salt is a chloride salt.

9. The method of claim 1, wherein the PNPase inhibitor and/or a PNPase purine nucleoside substrate is administered orally, intravenously, into the eye, or on the conjunctiva of the subject.

10. The method of claim 9, wherein administering comprises delivering the PNPase inhibitor and/or the PNPase purine nucleoside substrate into the eye of the subject.

11. The method of claim 10, wherein administering comprises repeated delivering to eye the subject.

12. The method of claim 1, wherein the PNPase inhibitor is the guanine comprising a substituent at the 8-position or the guanosine comprising a substituent at the 8-position.

13. The method of claim 1, wherein the age-related retinal disease is age-related macular degeneration (AMD), ganglion cell degeneration, glaucoma, Leber congenital amaurosis (LCA), retinitis pigmentosa, cone rod dystrophy, retinal detachment, hypertensive retinopathy, retinal vein occlusion (RVO), central retinal artery occlusion (CRAO), branch retinal artery occlusion (BRAO), or diabetic retinopathy.

14. The method of claim 13, wherein the subject has the AMD.

15. The method of claim 13, wherein the subject has glaucoma.

16. The method of claim 1, wherein administering to the subject a therapeutically effective amount of a PNPase inhibitor or a PNPase purine nucleoside substrate produces a decrease in at least one inflammatory cytokine.

17. The method of claim 16, wherein the at least one inflammation-associated cytokine comprises interleukin 1 beta (IL-1beta) or monocyte chemoattractant protein-1 (MCP-1).

18. The method of claim 1, wherein the subject is a veterinary subject.

19. The method of claim 1, wherein the subject is a human subject.

20. The method of claim 19, wherein the human subject is greater than 50 years of age.

21. The method of claim 20, wherein the human subject is greater than 60 years of age.
